(19) United States
(12) Patent Application Publication
(10) Pub. No.: US 2024/0181256 A1
(43) Pub. Date: Jun. 6, 2024

(54) VAGAL NERVE STIMULATION

(71) Applicants: Cochlear Limited, Macquarie University, NSW (AU); New York University (NYU), New York, NY (US)

(72) Inventors: Peter GIBSON, South Coogee, NSW (AU); Shawn ENTER, Eden Prairie, MN (US); Paul M. CARTER, Galston, NSW (AU); J. Thomas ROLAND, JR., Bronx, NY (US)

(21) Appl. No.: 18/569,107

(22) PCT Filed: Jun. 13, 2022
(86) PCT No.: PCT/IB2022/055447

§ 371 Date: Dec. 11, 2023

Related U.S. Application Data
(60) Provisional application No. 63/212,294, filed on Jun. 18, 2021.

(51) Int. Cl. A61N 1/36; A61N 1/05

(52) U.S. Cl. CPC A61N 1/36053; A61N 1/0558

(57) ABSTRACT

Presented herein are techniques in which a specific branch of the vagal nerve that extends to the car, referred to as the auricular branch of the vagal nerve (ABVN), is stimulated via an implantable stimulator. Described herein are implantable stimulation assembly arrangements for implantation adjacent to the auricular branch of the vagal nerve, and use of the implantable stimulating assembly arrangements in order to electrically stimulate the auricular branch of the vagal nerve.

### BACKGROUND

### Field of the Invention

The present subject matter relates generally to stimulation of the auricular branch of the vagal nerve.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: positioning at least one vagal nerve stimulation assembly within a recipient adjacent to soft tissue inclusive of at least one auricular branch of a vagal nerve of the recipient; and electrically stimulating the at least one auricular branch of the vagal nerve of the recipient via the least one vagal nerve stimulation assembly.

In another aspect, an apparatus is provided. The apparatus comprises: at least one vagal nerve stimulation assembly configured to be implanted within a recipient adjacent to a distal surface of soft tissue inclusive of at least one auricular branch of a vagal nerve of the recipient, wherein the at least one vagal nerve stimulation assembly comprises one or more implantable electrodes facing the distal surface of the soft tissue inclusive of the at least one auricular branch of the vagal nerve; an implantable module comprising a stimulator unit; and a lead region electrically connecting the stimulator unit to the at least one vagal nerve stimulation assembly.

In another aspect, a method is provided. The method comprises: opening a surgical incision behind an outer ear of a recipient; implanting a vagal nerve stimulation assembly into the recipient via the surgical incision; securing the at least one vagal nerve stimulation assembly within the recipient adjacent to tissue containing at least one auricular branch of the vagal nerve of the recipient; and closing the surgical incision with the vagal nerve stimulation assembly within the recipient.

### BRIEF DESCRIPTION OF THE DRAWINGS

Embodiments are described herein in conjunction with the accompanying drawings, in which:

FIG. 1A is a schematic diagram illustrating a side-view of an outer ear of a recipient of a vagal nerve stimulation system, in accordance with certain embodiments presented herein;

FIG. 1B is a schematic diagram illustrates a rear-view of the outer ear of FIG. 1A;

FIG. 2A is a line drawing representing the anatomy at a left ear of a recipient during a mastoidectomy prepared for cochlear implant surgery, in accordance with certain embodiments presented herein;

FIG. 2B is an image of a mastoidectomy prepared for cochlear implant surgery in a left ear of a recipient, corresponding to the schematic line drawing of FIG. 2A, in in accordance with certain embodiments presented herein;

FIG. 2C illustrates the image of FIG. 2B with the line drawing of FIG. 2A superimposed thereon;

FIG. 3A is a schematic top-view of an implantable vagal nerve stimulator shown with the line drawing of FIG. 2A and the associated anatomical structures, in accordance with certain embodiments presented herein;

FIG. 3B is a schematic side-view of the implantable vagal nerve stimulator of FIG. 3A during surgical placement, in accordance with certain embodiments presented herein;

FIG. 3C is a schematic side-view of the implantable vagal nerve stimulator of FIG. 3A following surgical placement, in accordance with certain embodiments presented herein;

FIG. 4A is a schematic cross-sectional view of a stimulation assembly associated with the implantable vagal nerve stimulator of FIG. 3A;

FIG. 4B is a top-view of the stimulation assembly associated with the implantable vagal nerve stimulator of FIG. 3A;

FIG. 5 is a schematic diagram illustrating a varying anatomical arrangement for the auricular branch of the vagal nerve through recipient tissue;

FIG. 6 is a schematic diagram illustrating the stimulation assembly associated with the implantable vagal nerve stimulator of FIG. 3A with the schematic tissue illustration of FIG. 5;

FIG. 7A is a cross-sectional side-view of a vagal nerve stimulation assembly, in accordance with certain embodiments presented herein;

FIG. 7B is a top-view of the vagal nerve stimulation assembly of FIG. 7A;

FIG. 7C illustrates the vagal nerve stimulation assembly of FIG. 7A with the schematic tissue illustration of FIG. 5;

FIG. 8A is a cross-sectional side-view of another vagal nerve stimulation assembly, in accordance with certain embodiments presented herein;

FIG. 8B is a top-view of the vagal nerve stimulation assembly of FIG. 8A;

FIG. 9A is top-view of another vagal nerve stimulation assembly, in accordance with certain embodiments presented herein;

FIG. 9B is a side-view of the vagal nerve stimulation assembly of FIG. 9A;

FIG. 10 is top-view of another vagal nerve stimulation assembly, in accordance with certain embodiments presented herein;

FIG. 11 is top-view of another vagal nerve stimulation assembly, in accordance with certain embodiments presented herein;

FIG. 12A is top-view of another vagal nerve stimulation assembly, in accordance with certain embodiments presented herein;

FIG. 12B is a side-view of the vagal nerve stimulation assembly of FIG. 12A;

FIG. 13 is top-view of another vagal nerve stimulation assembly, in accordance with certain embodiments presented herein;

FIG. 14 is top-view of another vagal nerve stimulation assembly, in accordance with certain embodiments presented herein;

FIG. 15 is a schematic diagram illustrating an example stimulation assembly support structure, in accordance with certain embodiments presented herein;

FIG. 16A is a schematic top-view of an implantable vagal nerve stimulator during surgical placement, in accordance with certain embodiments presented herein;

FIG. 16B is a schematic side-view of the implantable vagal nerve stimulator of FIG. 16A, during surgical placement;

FIG. 16C is a cross-sectional view of a vagal nerve stimulation assembly associated with the implantable vagal nerve stimulator of FIG. 16A;

FIG. 16D is a top-view of a vagal nerve stimulation assembly shown in FIG. 16C;

FIG. 17 is a schematic diagram illustrating an embodiment comprising two vagal nerve stimulation assemblies, in accordance with certain embodiments presented herein;

FIG. 18 is a block diagram of an example combined cochlear implant and vagal nerve stimulator, in accordance with certain embodiments presented;

FIG. 19 is a block diagram of an example combined vestibular implant and vagal nerve stimulator, in accordance with certain embodiments presented;

FIG. 20 is a flowchart of an example method, in accordance with embodiments presented herein;

FIG. 21 is a flowchart of another example method, in accordance with embodiments presented herein; and

FIG. 22 is a schematic diagram illustrate the main soft tissue layers at a location for implantation of a vagal nerve stimulation assembly, in accordance with embodiments presented herein.

### DETAILED DESCRIPTION

Vagal (vagus) nerve stimulation is in research for treatment of a wide variety of diseases, including epilepsy, depression, bipolar disorder, etc. Presented herein are techniques in which a specific branch of the vagal nerve that extends to the ear, referred to as the auricular branch of the vagal nerve (ABVN), is stimulated via an implantable stimulator. Described herein are implantable stimulation assembly arrangements for implantation adjacent to the auricular branch of the vagal nerve, and use of the implantable stimulating assembly arrangements in order to electrically stimulate the auricular branch of the vagal nerve. Also described herein are techniques for securing/fixing the implantable stimulating assembly arrangements adjacent the auricular branch of the vagal nerve, as well as techniques to optimize the electrical stimulation delivered to the recipient. Finally, presented herein are techniques for use of implantable auricular vagal nerve stimulation with other implantable medical functions.

Merely for ease of description, the techniques presented herein are primarily described herein with reference to vagal nerve stimulation alone, or in combination with a cochlear implant or vestibular implant. It is to be appreciated that the techniques presented herein may also be used with a variety of other implantable medical devices. For example, the techniques presented herein may be used with other hearing devices, including combinations of any of a cochlear implant, middle ear auditory prosthesis (middle ear implant), bone conduction device, direct acoustic stimulator, electro-acoustic prosthesis, auditory brain stimulator systems, etc. The techniques presented herein may also be used with devices that comprise or include tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

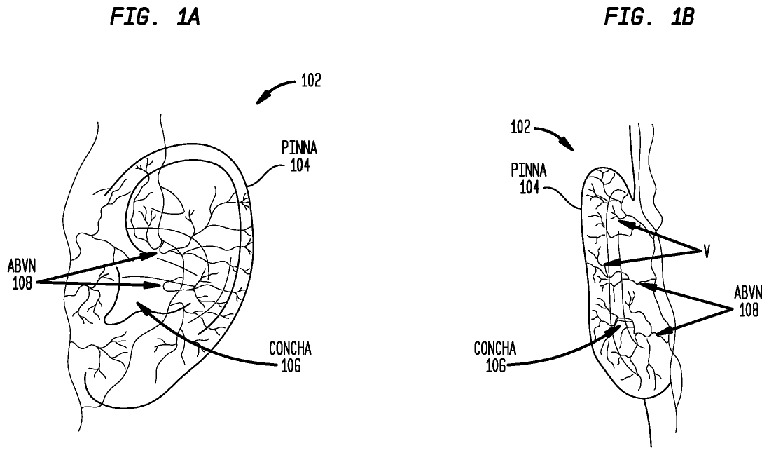

FIG. 1A is side-view of the outer ear 102 of a recipient, while FIG. 1B is a rear-view of the outer ear 102 of the recipient. The outer ear 102 includes, among other structures, the auricle (pinna) 104 and the concha (concha tissue) 106. The auricular branch of the vagal nerve (ABVN) 108 supplies sensory innervation to the skin of the ear canal, tragus, and the auricle 104. As shown in FIGS. 1A and 1B, the auricular branch of the vagal nerve 108 is surgically accessible from underneath the auricle 104 and passes through the concha 106. In particular, by elevating the auricle 104 posterior to the external auditory canal, the area under the concha 106 is exposed, whereby the auricular branch of the vagal nerve 108 can be readily accessed.

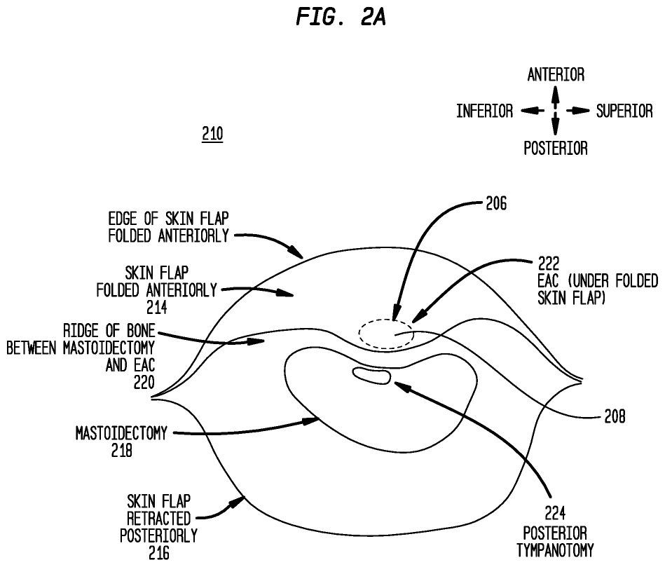

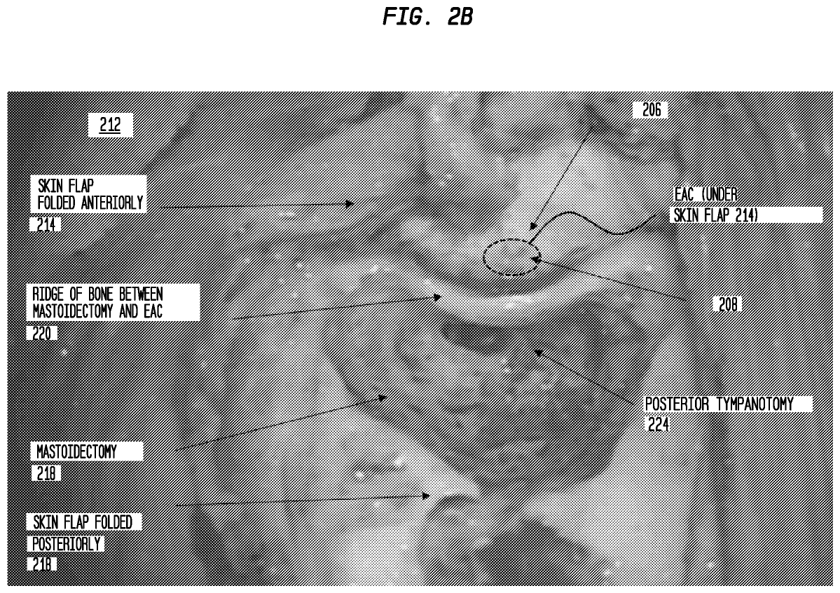

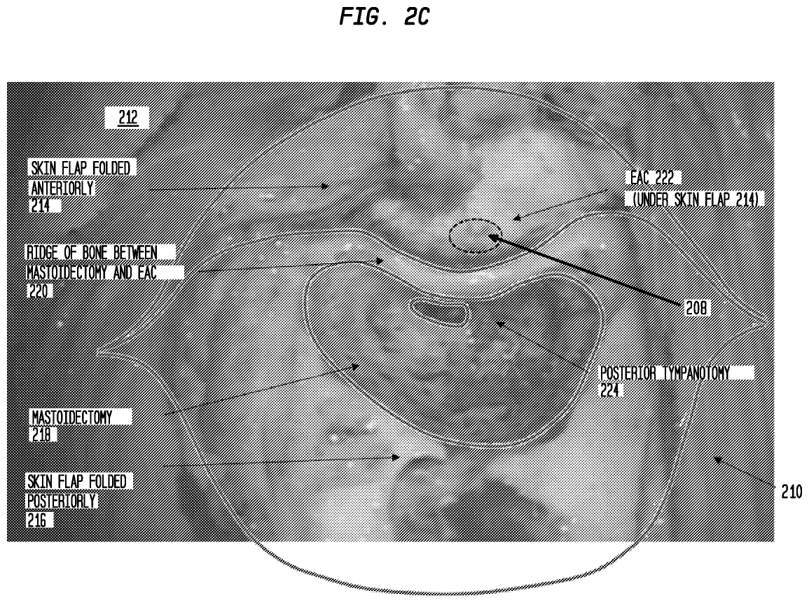

FIGS. 2A-2C illustrate further details relating to surgical access to the auricular branch of the vagal nerve 108. More specifically, FIG. 2A is a line drawing 210 representing the anatomy at a left ear of a recipient during a mastoidectomy prepared for a cochlear implant surgery. That is, FIG. 2A schematic illustrates an opening/cavity (mastoidectomy cavity or mastoidectomy) formed in the head of a recipient, at the recipient's ear, during a cochlear implant surgery. FIG. 2B is an image 212 of a mastoidectomy prepared for cochlear implant surgery in a left ear of a recipient, corresponding to the schematic line drawing of FIG. 2A. Finally, FIG. 2C illustrates the image 212 of FIG. 2B, with the line drawing 210 of FIG. 2A superimposed thereon. The same reference numbers are shown in FIGS. 2A, 2B, and 2C in order illustrate the relationship between the line drawing 210 and the image 212.

Shown in FIGS. 2A-2C is a first tissue flap 214 (e.g., skin and underlying tissue) that is, for example, folded anteriorly (e.g., pinna elevated from the bone and folded anteriorly) and a second tissue flap 216, that is, for example, retracted in a concertina style. The tissue flaps 214/216 are formed by making a surgical incision in the skin behind the concha (not shown in FIGS. 2A-2C). The first tissue flap 214 generally includes the concha of the recipient, which is adjacent the external auditory canal (EAC) 222.

The folding of the tissue flaps 214/216 provides access to the recipient's skull bone, in which a mastoidectomy 218 is formed. The mastoidectomy 218 is a surgical formed cavity in the skull bone that provides a surgeon access to, for example, the inner ear of the recipient. Also shown in FIGS. 2A-2C is a bone ridge 220 between mastoidectomy 218 and the external auditory canal 222 (located under tissue flap 214) and the posterior tympanotomy 224.

The auricular branch 208 of the vagal nerve follows a path that includes the EAC 222, the concha 206, and parts of the outer ear 102, inclusive of the pinna 104. For example, a portion of the auricular branch 208 of the vagal nerve is located inside the tissue flap 214 that is folded anteriorly (e.g., in an area under the EAC 222). For ease of description, the tissue surrounding the auricular branch 208 of the vagal nerve, namely the sections of the EAC 222, the concha 206, and the outer ear 102 are collectively and generally referred to in herein as “soft tissues inclusive of the auricular branch of the vagal nerve (STABVN)” or “AVBN soft tissues.” All of the soft tissues inclusive of the auricular branch of the vagal nerve can be stimulated by one or more of the techniques disclosed herein though small adjustments specific placement of the electrodes nearer to the other areas containing the auricular branch of the vagal nerve and/or extensions of the electrode array to ensure the targeted area is within the range of stimulation. For simplicity, this application will refer to placement of electrodes adjacent to the soft tissues inclusive of the auricular branch of the vagal nerve (i.e., adjacent the STABVN).

As noted, FIGS. 2A-2C illustrate a mastoidectomy 218, which is present in cases in which an implantable vagal nerve stimulator, in accordance with embodiments presented herein, is used in combination with a cochlear implant or vestibular implant. However, it is to be appreciated that implantable vagal nerve stimulators presented herein can be implemented without a cochlear implant or a vestibular implant. In such cases, a full mastoidectomy may not be required. Merely for ease of description, implantable vagal nerve stimulators presented herein are generally described with reference to the presence of a mastoidectomy and in combination cochlear implant or a vestibular implant, as such combinations illustrate additional considerations as compared to implantable vagal nerve stimulators alone. Moreover, there may be advantages in providing a device that can stimulate the vagal nerve in combination with the cochlear nerve and/or the vestibular organs.

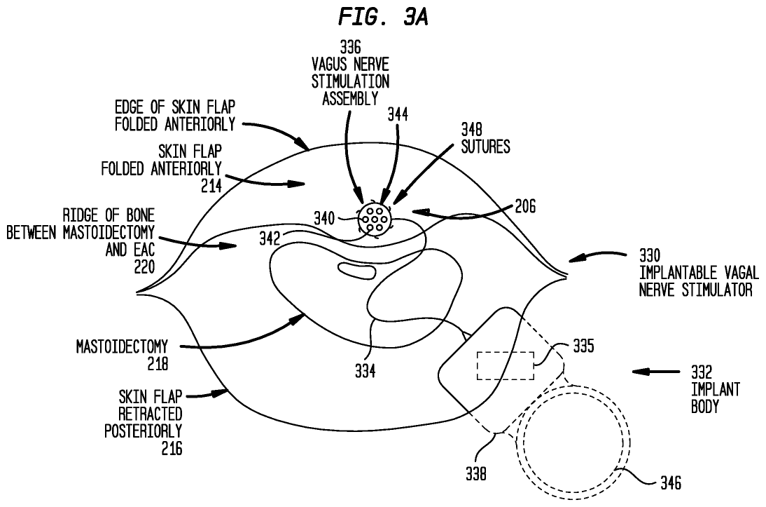

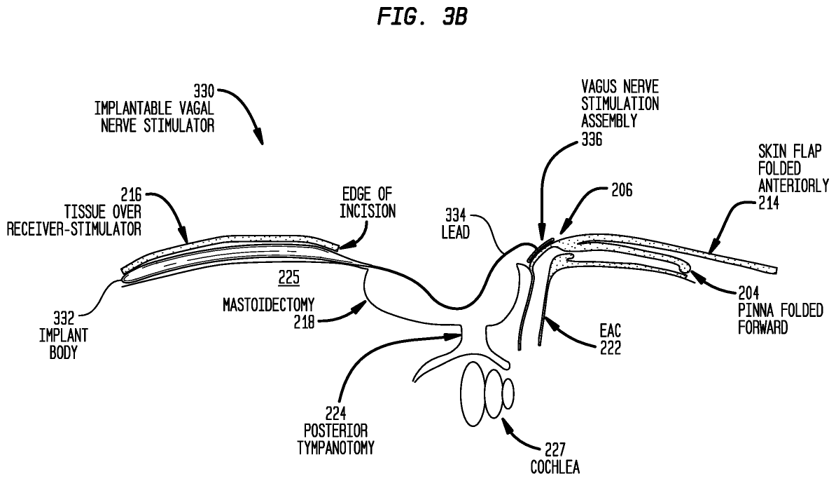

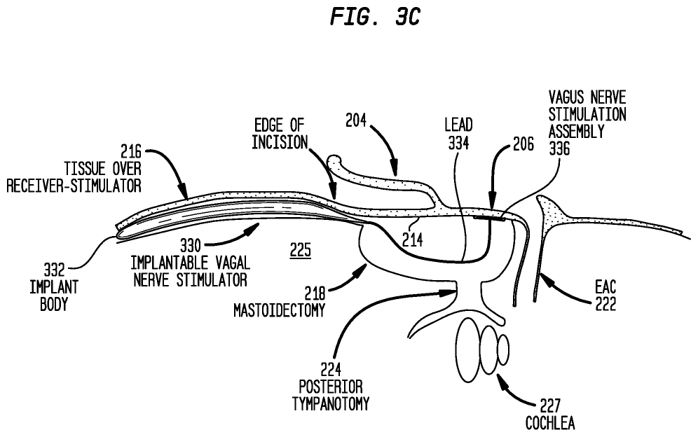

FIGS. 3A, 3B, and 3C are schematic diagrams illustrating an implantable vagal nerve stimulator 330, in accordance with embodiments presented herein. More specifically, FIG. 3A is a schematic top-view of the implantable vagal nerve stimulator 330 shown with the line drawing 210 of FIG. 2A and the associated anatomical structures shown therein. FIG. 3B is a schematic side-view of the implantable vagal nerve stimulator 330 during surgical placement (e.g., during surgery), while FIG. 3C is a schematic side-view of the implantable vagal nerve stimulator 330 following surgical placement (e.g., after surgery). FIGS. 3B and 3C represent a cross-section of a left side of the head of recipient, where, for ease of illustration, anatomical structures are shown in a simplified form.

In certain embodiments, the implantable vagal nerve stimulator 330 is configured to operate with one or more external components that provide, for example, power and/or data to the implantable vagal nerve stimulator 330. In other embodiments, the implantable vagal nerve stimulator 330 can be a totally implantable component having the ability to operate, for at least finite periods of time, without an external component. In such embodiments, an external component/device can be provided to, for example, periodically charge a battery of the totally implantable vagal nerve stimulator 330. In any event, for ease of illustration, FIG. 3A illustrates the implantable vagal nerve stimulator 330 without any external component.

As shown in FIGS. 3A-3C, the implantable vagal nerve stimulator 330 comprises an implant body (main module) 332, a lead region 334, and a vagal nerve stimulation assembly 336, all configured to be implanted under the skin/tissue (e.g., under/below tissue flaps 214 and 216) of the recipient. The implant body 332 generally comprises a hermetically-sealed housing 338 in a stimulator unit 335 is disposed. In addition to the stimulator unit 335, one or more other functional or electrical components can, depending on the arrangement and use of the implantable vagal nerve stimulator 330, also be disposed in the housing 338. These other components can include, for example, one or more rechargeable batteries, one or more sensors, one or more processors, memory, radio-frequency (RF) interface circuitry, etc. As shown, the implant body 332 can also include an internal/implantable coil 346 that is, for example, external to the housing 338, but which is connected to the RF interface circuitry via a hermetic feedthrough.

The vagal nerve stimulation assembly 336 is configured to be implanted in the recipient adjacent to the underside of the concha 206. Vagal nerve stimulation assembly 336 is formed by an electrically non-conductive (insulating) carrier member 340 and a plurality of conductive electrodes 342 disposed in (in or on) the carrier member 340. The plurality of electrodes collectively form an electrode array 344. In the examples of FIGS. 3A-3C, the stimulation assembly 336 is secured/attached to a portion of the tissue flap 214 (e.g., the concha tissue) via sutures/stitches 348. However, it is to be appreciated that the stimulation assembly 336 could be secured to the tissue flap 214 and/or the recipient's bone in other manners, such as with a biocompatible adhesive, etc.

The electrode array 344 is electrically connected to the stimulator unit 335 in the implant body 332 via lead region 334 and a hermetic feedthrough (not shown in FIG. 3A). Lead region 334 includes a plurality of conductors (wires) that electrically couple the electrodes 342 to the stimulator unit 335. In the embodiments of FIGS. 3A-3C, the implant body 332 is implanted underneath the tissue flap 216. However, it is to be appreciated that the implant body 332 could be implanted at other locations that could be, for example, farther from the vagal nerve stimulation assembly 336. In FIG. 3A, after suturing the vagal nerve stimulation assembly 336 to the tissue flap 214 over the site of the auricular branch of the vagal nerve, the pinna is folded back over and the spare lead region 334 rests in the mastoidectomy 218.

In FIG. 3A, the electrodes 342 are shown using dashed lines to indicate the electrodes 342 are implanted to face a portion of the tissue flap 214, such as underside of the concha 206. This is more clearly shown in FIGS. 3B and 3C, where, as noted, FIG. 3B shows the implantable vagal nerve stimulator 330 during surgery and FIG. 3C shows the implantable vagal nerve stimulator 330 following surgery. FIG. 3B illustrates the recipient's pinna 204 and concha 206 are folded forward, and that the electrodes 342 are positioned adjacent (e.g., abutting) the underside of the concha 206. Also shown is the recipient EAC 222 and the cochlea 227.

As shown in FIG. 3C, following surgery, the electrodes 342 of the vagal nerve stimulation assembly 336 face outward from the skull bone 225 and the cochlea 227. That is, as noted, the vagal nerve stimulation assembly 336 is sutured to the underside of the tissue 214 such that the electrode array 344 is adjacent the underside of the concha 206 in the area of the auricular branch of the vagal nerve (while the skin flap with the pinna is folded anteriorly).

In operation, the stimulator unit 335 in the implant body 332 is configured to generate electrical stimulation signals (current signals) that are delivered to the recipient via the electrode array 344. As a result, the vagal nerve stimulator 330 is configured to electrically stimulate the auricular branch of the vagal nerve from an implanted location. Various configurations to the vagal nerve stimulator 330 can be made by, for example, activating and/or deactivating specific electrodes 342 in the electrode arry 344, setting attributes of the electrical stimulation signals, etc., to customize the stimulation of the auricular branch of the vagal nerve to the needs of the specific recipient.

For ease of illustration, FIGS. 3A-3C illustrate only the vagal nerve stimulation assembly 336. As noted, vagal nerve stimulation can be combined with, for example, cochlear stimulation, vestibular stimulation, or other implantable medical devices. Moreover, it is also to be appreciated that, with many devices now having connectivity to external components and to each-other, there is the potential to synchronize the vagal nerve stimulation described herein with other types of therapies, monitoring, etc. with a range of devices that are distributed at various locations within the body of the recipient, worn by recipient, carried by the recipient, etc. For example an epilepsy monitoring and/or stimulation device monitoring could be a separate electronic module and send a control message to the vagal stimulator to start operation or moderate stimulus based on the monitoring of the epilepsy condition. A similar combination may be used with heart treatment devices, seizure devices, and/or devices configured to treat other conditions. In another example, a vestibular therapy device could be on the opposite ear to avoid having too much physical structure on the one side of the head, which can be impractical and a challenge in small heads or thin skin of older people. Moreover, as described further below, the same implant body 332 can function to deliver stimulation signals via multiple different stimulation assemblies. It is to be appreciated that these are merely examples of the range of possibilities for combining the use of stimulation of the auricular branch 208 of the vagal nerve with any of a number of different types of treatments.

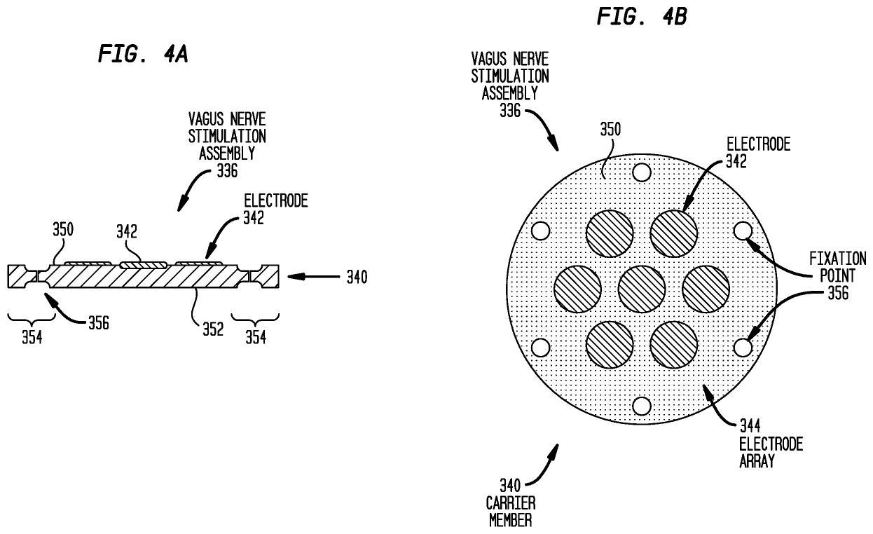

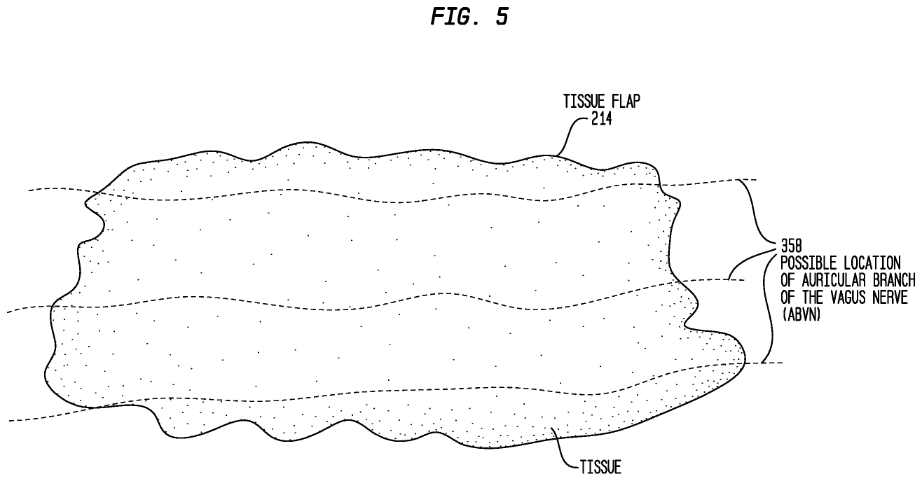

FIG. 4A illustrates a cross-sectional view of the stimulation assembly 336 of FIGS. 3A-3C, while FIG. 4B is a top-view of the stimulation assembly 336. As shown, the carrier member 340 of the stimulation assembly 336 has a generally planar and circular shape and is formed from an insulating (non-conductive) material (e.g., silicone). The carrier member 340 is relatively thin such that it can conform to the tissue of the concha 206, but it is also robust for reliability.

As noted, the vagal nerve stimulation assembly 336 includes an electrode array 344 that is formed by a plurality of electrodes 342. As described further below, each of the plurality of electrodes 342 can be independently activated or deactivated to select the electrode(s) for stimulation of the vagal nerve and avoid stimulation of other nerves in the region. The plurality of electrodes 342 are disposed on or at a first (tissue facing) surface 350 of the carrier member that, as noted, is implanted so as to face/abut the concha 206. The carrier member 340 includes a second surface 352 of the carrier member 340 (shown in FIG. 4A) is formed by the insulating material.

In certain examples, the vagal nerve stimulation assembly 336 can be configured to deliver one or more therapeutic substances to the recipient. For example, the carrier member 340 and/or the electrodes 342 can be loaded/doped with, and/or coated with, one or more therapeutic substances, such as dexamethasone to moderate the tissue growth around the electrode array after surgery, which in turn can provide the benefit of minimizing impedance and power consumption.

The carrier member 340 includes an inert border/margin 354 that, in this example extends around the electrode array 344 (e.g., around the outer circumference of the carrier member) for use in fixing/securing the stimulation assembly to soft tissues inclusive of the auricular branch of the vagal nerve, such as the underside of the tissue flap 214. In this example, the inert margin 354 includes fixation points 356, which comprise integrated mechanical weaknesses in the carrier member material that enable a surgeon to suture or screw the vagal nerve stimulation assembly 336 to the tissue flap 214, bone, etc. The fixation points 356 can comprise, for example, pre-formed apertures in the carrier member 340, relatively thinner sections of the carrier member 340, etc. In general, securing the vagal nerve stimulation assembly 336 to the tissue flap 214 ensures that the vagal nerve stimulation assembly 336 remains in the intended position under the auricular branch of the vagal nerve. Without fixation, the vagal nerve stimulation assembly 336 could shift relative to the auricular branch of the vagal nerve, which could adversely impact the effectiveness of stimulation.

It is known that the auricular branch of the vagal nerve is known to pass through the tissue flap 214 of the recipient in a particular direction (e.g., in a posterior/anterior direction). However, the specific path of the auricular branch of the vagal nerve within the tissue flap is unknown and can vary for different recipients. This varying anatomical arrangement is schematically represented in FIG. 5, where the general direction of the auricular branch of the vagal nerve through the tissue flap 214 is shown as being left-to-right. However, the dashed lines 358 generally represent the possible specific paths of the auricular branch of the vagal nerve through the tissue flap 214.

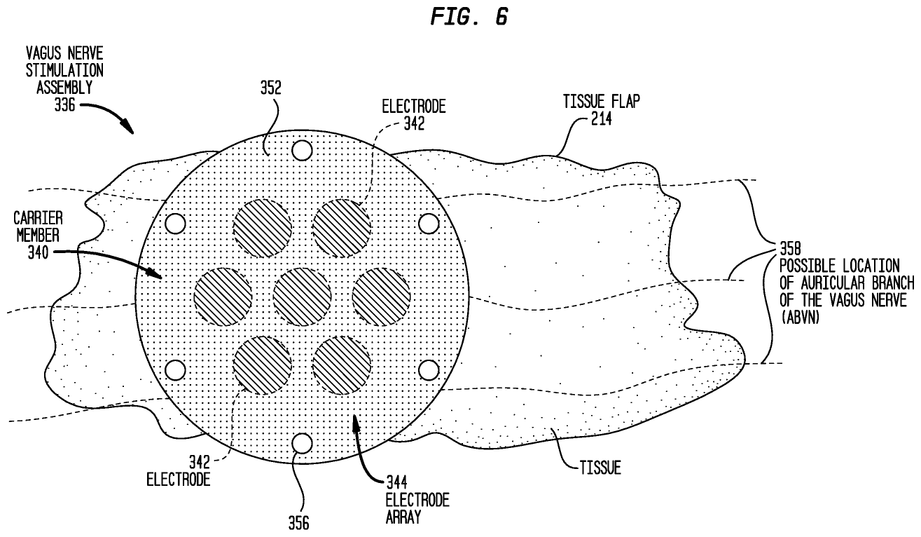

The techniques presented herein address the varying anatomical arrangement of the auricular branch of the vagal nerve within tissue flap 214 through the use of the plurality of independent electrodes that can be individually activated/deactivated. The ability to individually activate/deactivate the electrodes enables the selection of one or more specific electrodes for stimulation of the auricular branch of the vagal nerve and the ability to avoid or minimize stimulation of other nerves in the region. More specifically, shown in FIG. 6 is the vagal nerve stimulation assembly 336 with the schematic illustration of tissue flap 214 from FIG. 5. In this example, the second (insulating) surface 352 of the carrier member 340 is shown and the electrodes 342 face the tissue flap 214 and, as a result, would be obscured beneath the carrier member 340 in this particular view. However, merely for purposes of illustration, the electrodes 342 are shown in FIG. 6 using dash lines.

As shown in FIG. 6, the electrodes 342 of the electrode array 342 are sufficiently dispersed so as to cover the different specific paths of the auricular branch of the vagal nerve through the tissue flap 214. In accordance with embodiments presented herein, following implantation of the vagal nerve stimulation assembly 336 into the recipient, an electrode selection process is performed to determine which one or more of the electrodes 342 optimally stimulate the auricular branch of the vagal nerve. The one or more the electrodes 342 optimally stimulate the auricular branch of the vagal nerve are activated for subsequent use, while the remaining electrodes can be deactivated and/or activated in a manner so as to focus the electrical stimulation at a particular location (e.g., source or sink current to perform current focusing and/or current steering techniques). In general, the electrode selection process is used to identify the electrodes that most effectively stimulate the auricular branch of the vagal nerve (e.g., in terms of power cost, proximity, precision, etc.), while minimizing stimulation of non-target nerves (e.g., de-activate electrodes that are not effective or which stimulate other nerves that should not be stimulated).

In certain embodiments, it may be sufficient to select one or more electrodes and stimulate in monopolar mode (stimulation between an electrode on the vagal nerve stimulation assembly and a reference electrode on the device or a separate flying lead). However, it is also It is also noted that stimulation may be focused on the desired area of the auricular branch of the vagal nerve through a number of stimulation strategies. For example, it may be beneficial to stimulate between two or more selected electrodes on the vagal nerve stimulation assembly. It may also be beneficial to use a more sophisticated strategy of customizing the quantity and direction of current from a number of electrodes to focus the stimulation on a desired area.

In addition, it is noted that the choice of stimulation regime could be determined by the recipient and/or based on recipient feedback. For example, the recipient may be in the best position to determine the most beneficial stimulation electrodes, patterns, etc., and either control directly or to take note of what works and when (applying ecological momentary assessment methods). Controls could include, for example, choice of electrodes; stimulation levels; duration of stimulation; timing of stimulation (e.g. during the day or night; or whether at same time or different time to cochlear stimulation), etc.

The electrodes 342 of the electrode array 344 can have different sizes, shapes, spacing, etc. In certain embodiments, the gaps or spacing between the electrodes 342 is below a predetermined distance in order to reduce risk of missing the auricular branch of the vagal nerve during the electrode selection process.

As noted, FIGS. 4A, 4B, and 6 illustrate an embodiment in which the vagal nerve stimulation assembly 336 comprises a carrier member 340 having a round shape and an electrode array 344 having a general hexagonal pattern of electrodes 342. It is to be appreciated that these specific shapes/configurations are merely illustrative and many different carrier member shapes, electrode patterns, electrode sizes, and number of electrodes can be used in embodiments presented herein.

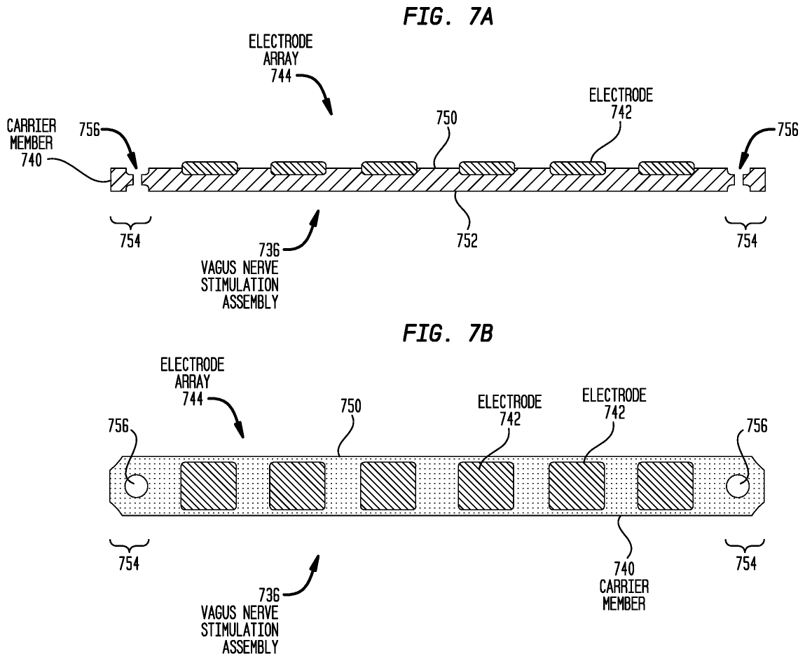

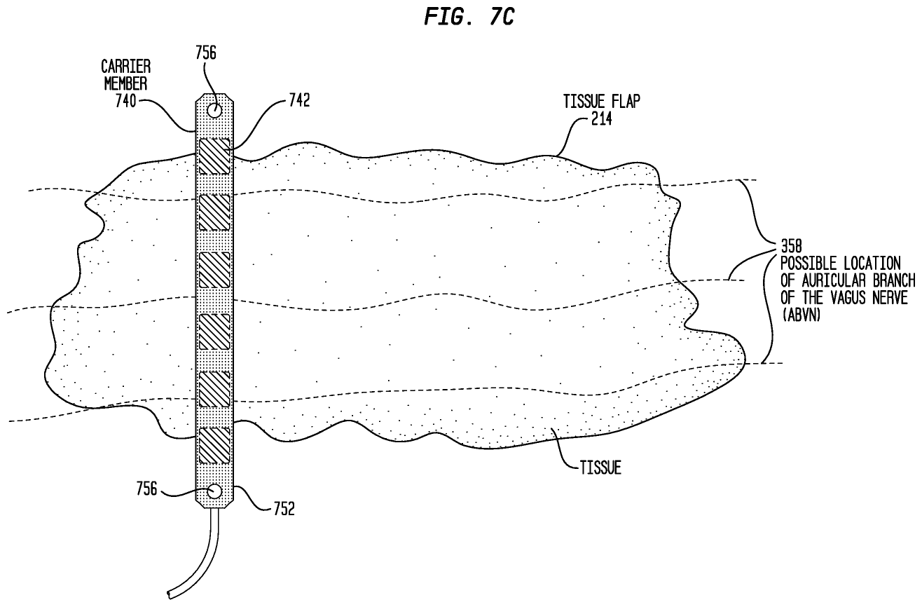

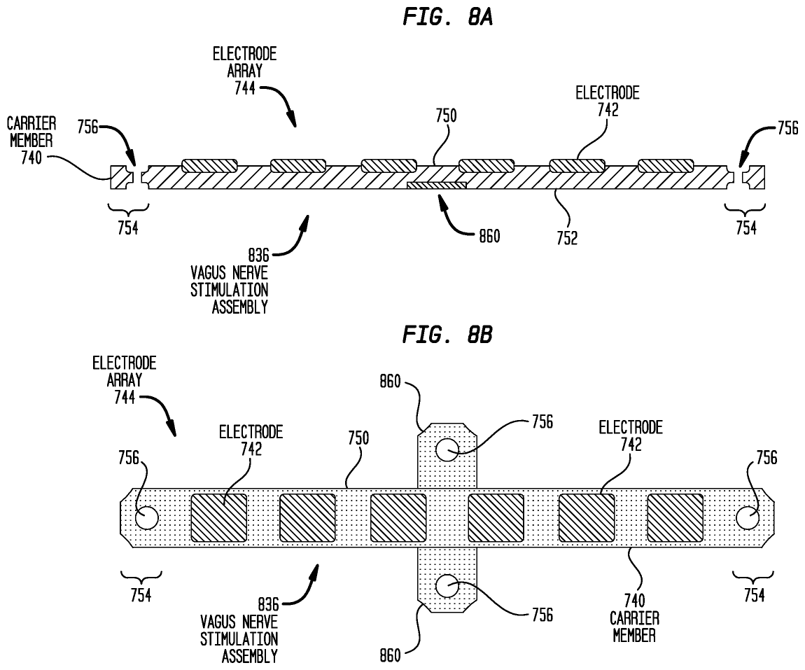

For example, FIGS. 7A, 7B, and 7C illustrate an example vagal nerve stimulation assembly 736, in accordance with certain embodiments presented herein. More specifically, FIG. 7A is cross-sectional side-view of the vagal nerve stimulation assembly 736, while FIG. 7B is top-view of the vagal nerve stimulation assembly 736. FIG. 7C illustrates the vagal nerve stimulation assembly 736 with the schematic illustration of tissue flap 214 from FIG. 5.

The vagal nerve stimulation assembly 736 comprises an elongate non-conductive (insulating) carrier member 740 and a plurality of conductive electrodes 742. In this example, the electrodes 742 are disposed in a linear pattern forming an elongate electrode array 744. The plurality of electrodes 742 are disposed on or at a first (tissue facing) surface 750 of the carrier member that is implanted so as to face/abut the tissue flap 214. The carrier member 740 includes a second surface 752 (shown in FIG. 7A) that is formed by the insulating material. The electrode array 744 is electrically connected to a stimulator unit, such as that shown in FIG. 3A.

Similar to the above embodiments, the vagal nerve stimulation assembly 736 can be configured to deliver one or more therapeutic substances to the recipient. For example, the carrier member 340 and/or the electrodes 342 the can be loaded/doped with, and/or coated with, one or more therapeutic substances, such as dexamethasone to moderate the tissue growth around the electrode array after surgery, which in turn can provide the benefit of minimizing impedance and power consumption.

The stimulation assembly 736 is configured to be secured/attached to, for example, a portion of the tissue flap 214 (FIG. 7C) and/or the recipient's bone (e.g., via sutures/stitches, screws, etc.) such that the electrodes 742 face the tissue flap. In this example, the carrier member 740 includes inert borders/margins 754 at both ends of the electrode array 744 for use in fixing/securing the stimulation assembly to the underside of the tissue flap 214 (e.g., the underside of the concha 206). In this example, the inert margins 754 include fixation points 756, which comprise integrated mechanical weaknesses in the carrier member material that enable a surgeon to suture the vagal nerve stimulation assembly 736 to the tissue flap 214. The fixation points 756 can comprise, for example, pre-formed apertures in the carrier member 740, relatively thinner sections of the carrier member 740, etc. In general, securing the vagal nerve stimulation assembly 736 to the tissue flap 214 ensures that the electrode array 744 remains in the intended position under the auricular branch of the vagal nerve. Without fixation, the electrode array 744 could shift relative to the auricular branch of the vagal nerve, which could adversely impact the effectiveness of stimulation. It is to be appreciated that the stimulation assembly 736 could also or alternatively be secured to the tissue flap in other manners, such as a biocompatible adhesive, etc.

As noted, FIG. 7C illustrates the vagal nerve stimulation assembly 736 with the schematic illustration of tissue flap 214 from FIG. 5. In this example, the second (insulating) side 752 of the carrier member 740 is shown and the electrodes 742 face the tissue flap 214 and, as a result, the electrodes would be obscured beneath the carrier member 740 in this particular view. However, merely for purposes of illustration, the electrodes 742 are shown in FIG. 7C using dash lines.

As shown in FIG. 7C, the electrodes 742 of the electrode array 744 are sufficiently dispersed so as to cover the different specific paths of the auricular branch of the vagal nerve through the tissue flap 214. In accordance with embodiments presented herein, following implantation of the vagal nerve stimulation assembly 736 into the recipient, an electrode selection process is performed to determine which one or more of the electrodes 742 optimally stimulate the auricular branch of the vagal nerve. The one or more the electrodes 742 optimally stimulate the auricular branch of the vagal nerve are activated for subsequent use, while the remaining electrodes can be deactivated and/or activated in a manner so as to focus the electrical stimulation at a particular location (e.g., source or sink current to perform current focusing and/or current steering techniques). That is, the electrodes 342 can be independently be activated or deactivated to select the electrode(s) for stimulation of the vagal nerve and avoid stimulation of other nerves in the region.

It is noted that the perpendicular arrangement for the vagal nerve stimulation assembly 736 shown in FIG. 7C is merely illustrative. In alternative embodiments, the vagal nerve stimulation assembly 736 could alternatively be angled by various amounts and still effectively capture the auricular branch of the vagal nerve.

The electrodes 742 of the electrode array 744 can have different sizes, shapes, spacing, etc. In certain embodiments, the gaps or spacing between the electrodes 742 is below a predetermined distance in order to reduce risk of missing the auricular branch of the vagal nerve during the electrode selection process.

In the example of FIGS. 7A-7C, the elongate/linear vagal nerve stimulation assembly 736 could be fixed/secured to the ridge of bone under the concha (e.g., ridge 220 from FIGS. 2A-2C). Instead of, or in addition to use of sutures or screws, the vagal nerve stimulation assembly 736 could be secured using a biocompatible adhesive (e.g., fibrin glue adhesive).

FIGS. 7A-7C generally illustrate that the carrier member 740, and thus the vagal nerve stimulation assembly 736, has a generally rectangular shape with equally spaced. In alternative embodiments, the carrier member 740 could be partly curved, ovoid, or could be formed by a combination of shapes. Also, different electrode patterns and numbers of electrodes could be employed.

FIGS. 8A and 8B illustrate another example vagal nerve stimulation assembly 836, in accordance with certain embodiments presented herein. More specifically, FIG. 8A is cross-sectional side-view of the vagal nerve stimulation assembly 836, while FIG. 8B is top-view of the vagal nerve stimulation assembly 836.

The vagal nerve stimulation assembly 836 is similar to the vagal nerve stimulation assembly 736 of FIGS. 7A-7C and comprises the carrier member 740 and the plurality of electrodes 742 disposed in a linear pattern forming an elongate electrode array 744. Again, the plurality of electrodes 742 are disposed on or at a first (tissue facing) surface 750 of the carrier member that is implanted so as to face/abut the tissue flap 214 (not shown in FIGS. 8A and 8B). The carrier member 740 includes a second surface 752 (shown in FIG. 8A) that is formed by the insulating material. The electrode array 744 is electrically connected to a stimulator unit, such as that shown in FIG. 3A.

The vagal nerve stimulation assembly 836 is configured to be secured/attached to, for example, a portion of the tissue flap 214 (FIG. 7C) or bone of the recipient (e.g., via sutures/stitches, screws, etc.) such that the electrodes 742 face the tissue flap. In this example, the carrier member 740 includes inert borders/margins 754 at both ends of the electrode array 744 for use in fixing/securing the stimulation assembly to the underside of the tissue flap 214 (e.g., the underside of the concha 206). In this example, the inert margins 754 include fixation points 756, which comprise integrated mechanical weaknesses in the carrier member material that enable a surgeon to suture or screw the vagal nerve stimulation assembly 736 to the tissue flap 214 and/or to bone of the recipient. The fixation points 756 can comprise, for example, pre-formed apertures in the carrier member 740, relatively thinner sections of the carrier member 740, etc.

In addition to the above, the vagal nerve stimulation assembly 836 further includes two securement tabs 860 disposed at a mid-point of the carrier member 740. Each of these securement tabs 860, which extend longitudinally from the carrier member 740, include a respective fixation point 756 for use in further securing the vagal nerve stimulation assembly 836 to the tissue flap.

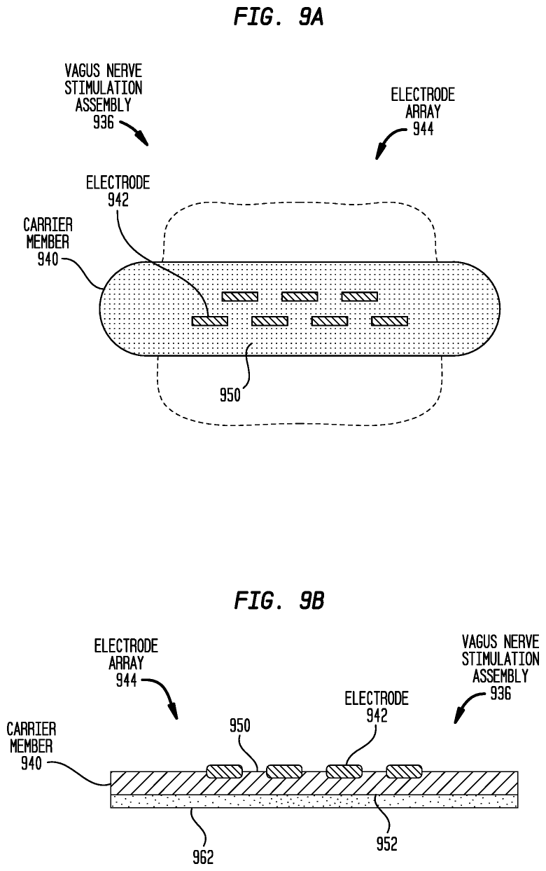

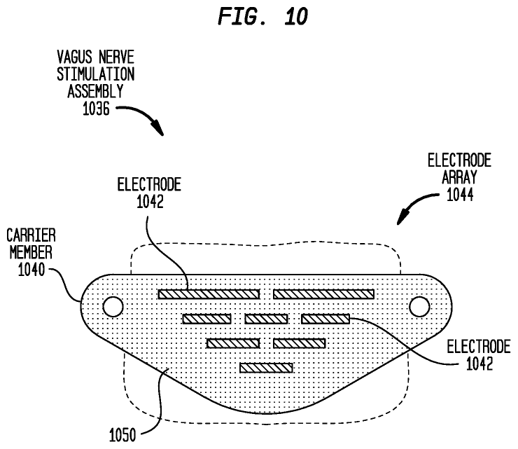

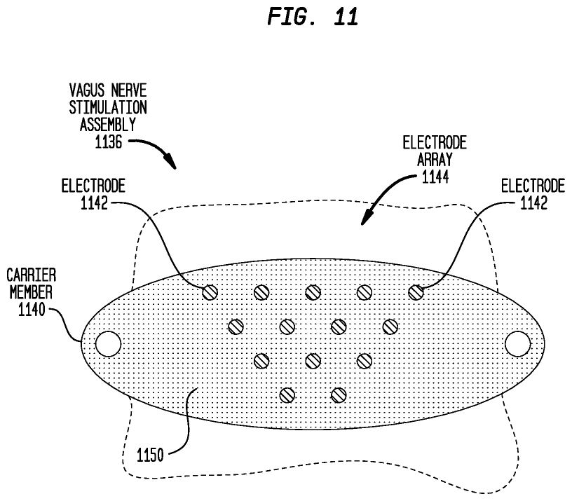

FIG. 9A is a top-view of another vagal nerve stimulation assembly 936, in accordance with certain embodiments presented herein, while FIG. 9B is a side-view of the vagal nerve stimulation assembly 936. In this example, the vagal nerve stimulation assembly 936 comprises an elongate non-conductive (insulating) carrier member 940 and a plurality of conductive electrodes 942. In this example, the electrodes 942 are disposed in a dual-row pattern (e.g., two elongate rows of electrodes) forming an electrode array 944.

As described elsewhere herein, each of the plurality of electrodes 942 can be independently be activated or deactivated to select one or more of electrode(s) for stimulation of the auricular branch of vagal nerve and to avoid stimulation of other nerves in the region. The electrodes 942 of the electrode array 944 can have different sizes, shapes, spacing, etc. In certain embodiments, the gaps or spacing between the electrodes 942 is below a predetermined distance in order to reduce risk of missing the auricular branch of the vagal nerve during the electrode selection process.

The plurality of electrodes 942 are disposed on or at a first (tissue facing) surface 950 of the carrier member that is implanted so as to face/abut soft tissues inclusive of the auricular branch of the vagal nerve, such as the tissue flap 214 (not shown in FIGS. 9A and 9B). The carrier member 940 includes a second surface 952 (shown in FIG. 9B) that is formed by the insulating material. The electrode array 944 is electrically connected to a stimulator unit, such as that shown in FIG. 3A.

The vagal nerve stimulation assembly 936 is configured to be secured/attached to a portion of the soft tissues inclusive of the auricular branch of the vagal nerve via a biocompatible adhesive 962. The biocompatible adhesive 962 is configured to be attached to, or integrated with, the second surface 952. It is to be appreciated that the vagal nerve stimulation assembly 936 could be adhered to the tissue flap in alternative manners, such as via sutures/stitches, etc.

FIG. 10 is a top-view of another vagal nerve stimulation assembly 1036, in accordance with certain embodiments presented herein. In this example, the vagal nerve stimulation assembly 1036 comprises an elongate non-conductive (insulating) carrier member 1040 and a plurality of conductive electrodes 1042. In this example, the electrodes 1042 are disposed in a multiple-row pattern (e.g., a plurality of rows of electrodes) forming an electrode array 1044. The electrode array 1044 specifically comprises four (4) rows in the example of FIG. 10, but other arrangements are possible. In addition, in order to accommodate the four rows of electrodes 1042, the carrier member 1040 has a shape in which the carrier member is wider in a central region, but which has tapered ends (e.g., a general triangular or trapezoidal shape).

As described elsewhere herein, each of the plurality of electrodes 1042 can be independently be activated or deactivated to select one or more of electrode(s) for stimulation of the auricular branch of vagal nerve and to avoid stimulation of other nerves in the region. The electrodes 1042 of the electrode array 1044 can have different sizes, shapes, spacing, etc. In certain embodiments, the longitudinal and/or lateral gaps/spacing between the electrodes 1042 is below a predetermined distance in order to reduce risk of missing the auricular branch of the vagal nerve during the electrode selection process.

The plurality of electrodes 1042 are disposed on or at a first (tissue facing) surface 1050 of the carrier member that is implanted so as to face/abut soft tissues inclusive of the auricular branch of the vagal nerve, such as the tissue flap 214 (not shown in FIG. 10). The carrier member 1040 includes a second surface (also not shown in FIG. 10) that is formed by the insulating material. The electrode array 1044 is electrically connected to a stimulator unit, such as that shown in FIG. 3A. The vagal nerve stimulation assembly 1036 is configured to be secured/attached to a portion of the soft tissues inclusive of the auricular branch of the vagal nerve via, for example, a biocompatible adhesive, via sutures/stitches, combinations thereof, etc.

FIG. 11 is a top-view of another vagal nerve stimulation assembly 1136, in accordance with certain embodiments presented herein. In this example, the vagal nerve stimulation assembly 1136 comprises an elongate non-conductive (insulating) carrier member 1140 and a plurality of conductive electrodes 1142. In this example, the electrodes 1142 comprise a form of a distributed “dot-matrix” (e.g., a plurality of micro-electrodes distributed across the surface 1150 of the carrier member 1140).

As described elsewhere herein, each of the plurality of electrodes 1142 can be independently be activated or deactivated to select one or more of electrode(s) for stimulation of the auricular branch of vagal nerve and to avoid stimulation of other nerves in the region. The electrodes 1142 of the electrode array 1144 can have different sizes, shapes, arrangements, spacing, etc. In certain embodiments, the longitudinal and/or lateral gaps/spacing between the electrodes 1142 is below a predetermined distance in order to reduce risk of missing the auricular branch of the vagal nerve during the electrode selection process.

The plurality of electrodes 1142 are disposed on or at a first (tissue facing) surface 1150 of the carrier member that is implanted so as to face/abut soft tissues inclusive of the auricular branch of the vagal nerve, such as the tissue flap 214 (not shown in FIG. 11). The carrier member 1140 includes a second surface (also not shown in FIG. 11) that is formed by the insulating material. The electrode array 1144 is electrically connected to a stimulator unit, such as that shown in FIG. 3A. The vagal nerve stimulation assembly 1136 is configured to be secured/attached to a portion of the soft tissues inclusive of the auricular branch of the vagal nerve via, for example, a biocompatible adhesive, via sutures/stitches, combinations thereof, etc.

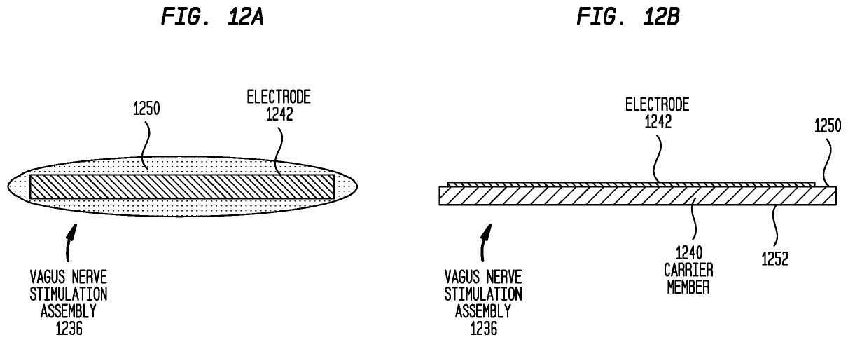

FIG. 12A is a top-view of another vagal nerve stimulation assembly 1236, in accordance with certain embodiments presented herein, while FIG. 12B is a cross-sectional view of the vagal nerve stimulation assembly 1236. In this example, the vagal nerve stimulation assembly 1236 comprises an elongate non-conductive (insulating) carrier member 1240 and a single elongate conductive electrode 1242. That is, in contrast to alternative embodiments, the electrode array is replaced by a single electrode that extends across the plurality of possible locations for the auricular branch of the vagal nerve in the tissue flap 214.

The electrode 1242 is disposed on or at a first (tissue facing) surface 1250 of the carrier member that is implanted so as to face/abut soft tissues inclusive of the auricular branch of the vagal nerve, such as the tissue flap 214 (not shown in FIGS. 12A or 12B). The carrier member 1240 includes a second surface 1252 (shown in FIG. 12B) that is formed by the insulating material. The electrode 1242 is electrically connected to a stimulator unit, such as that shown in FIG. 3A. The vagal nerve stimulation assembly 1236 is configured to be secured/attached to a portion of the soft tissues inclusive of the auricular branch of the vagal nerve via, for example, a biocompatible adhesive, via sutures/stitches, combinations thereof, etc.

As compared to the use of an electrode array, the example arrangement of FIGS. 12A and 12B can be easier to manufacturer and program (e.g., no electrode selection process). However, the use of a single electrode 1242 can require higher power to operate and has a higher risk of affecting nerves other than the auricular branch of the vagal nerve in the tissue flap 214.

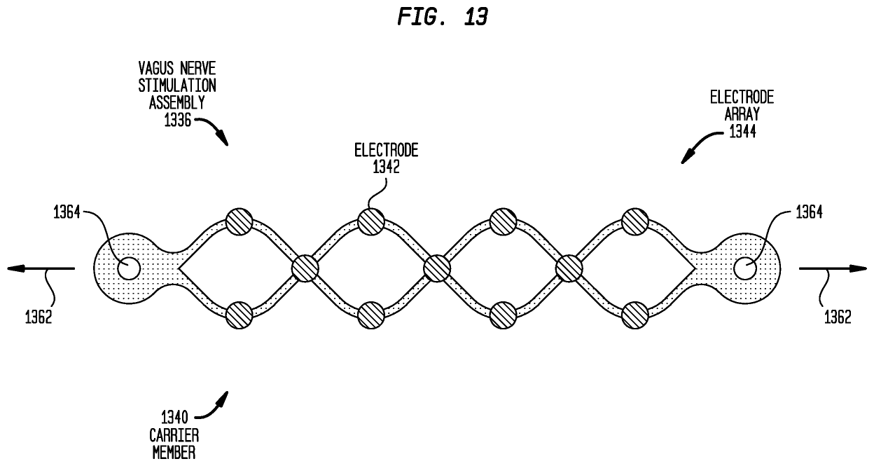

FIG. 13 is a top-view of another vagal nerve stimulation assembly 1336, in accordance with certain embodiments presented herein. In this example, the vagal nerve stimulation assembly 1336 comprises an elongate non-conductive (insulating) carrier member 1340 and a plurality of conductive electrodes 1342. In this example, the carrier member 1340 has a general lattice structure/frame and is formed from an resilient material that allows the carrier member 1340 to stretch/expand in a longitudinal direction, as represented by arrows 1362. That is, the longitudinal length of the carrier member 1340 is variable. The expandable lattice structure of the carrier member 1340 in FIG. 13 can be beneficial to, for example, to account for a variety of widths in different recipients.

Disposed at the opposing ends of the carrier member 1340 are fixation points 1364. The fixations points 1364 are configured to receive, for example, a screw (e.g., cortical screw), one or more sutures, etc., for securing the vagal nerve stimulation assembly 1336 to the recipient's tissue and/or bone. Additionally, the lattice structure of the carrier member 1340 can be sutured directly to adjacent tissue.

In the example of FIG. 13, the electrodes 1342 are distributed across the lattice of the carrier member 1340. As the longitudinal length of the carrier member 1340 is varied, the spacing between the electrodes 1342 varies.

As described elsewhere herein, each of the plurality of electrodes 1342 can be independently be activated or deactivated to select one or more of electrode(s) for stimulation of the auricular branch of vagal nerve and to avoid stimulation of other nerves in the region. The electrodes 1342 of the electrode array 1344 can have different sizes, shapes, arrangements, spacing, etc.

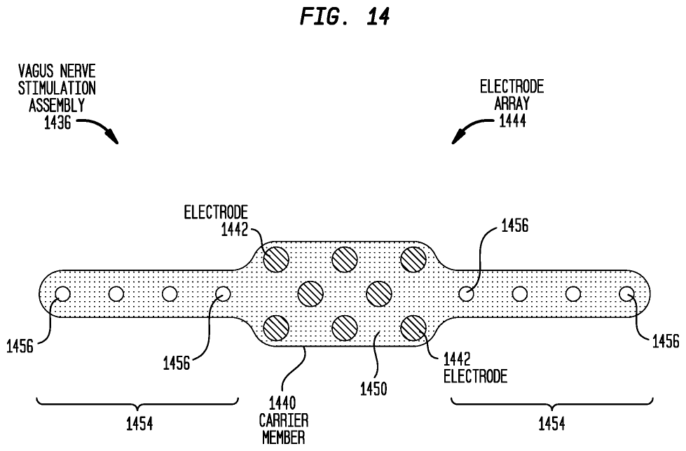

FIG. 14 is a top-view of yet another example vagal nerve stimulation assembly 1436, in accordance with certain embodiments presented herein. The vagal nerve stimulation assembly 1436 comprises an elongate non-conductive (insulating) carrier member 1440 and a plurality of conductive electrodes 1442. In this example, the electrodes 1442 are disposed in a distributed pattern forming an elongate electrode array 1444. The plurality of electrodes 1442 are disposed on or at a first (tissue facing) surface 1450 of the carrier member that is implanted so as to face/abut soft tissues inclusive of the auricular branch of the vagal nerve, such as the tissue flap 214 (not shown in FIG. 14). The carrier member 1440 includes a second surface (not shown in FIG. 14) that is formed by the insulating material. The electrode array 1444 is electrically connected to a stimulator unit, such as that shown in FIG. 3A.

The vagal nerve stimulation assembly 1436 is configured to be secured/attached to soft tissues inclusive of the auricular branch of the vagal nerve and/or bone adjacent to the tissue flap, such that the electrodes 1442 face the soft tissues inclusive of the auricular branch of the vagal nerve. As such, the carrier member 1440 includes inert margins 1454 at both ends of the electrode array 1444 for use in fixing/securing the stimulation assembly to the soft tissues inclusive of the auricular branch of the vagal nerve and/or to the recipient's bone. In this example, the inert margins 1454 each include a plurality of fixation points 1456, which comprise integrated mechanical weaknesses in the carrier member material that enable a surgeon to suture the vagal nerve stimulation assembly 1436 to the soft tissues inclusive of the auricular branch of the vagal nerve and/or to screw the vagal nerve stimulation assembly 1436 to the recipient's bone. The fixation points 1456 can comprise, for example, pre-formed apertures in the carrier member 1440, relatively thinner sections of the carrier member 1440, etc. In general, the presence of the plurality of fixation points 1456 allows a surgeon to select the fixation location that is best suited for recipient. As such, the arrangement of FIG. 14 can be beneficial to, for example, to account for a variety of widths in different recipients. If present, the surgeon can remove excess portions of the inert margins 1454.

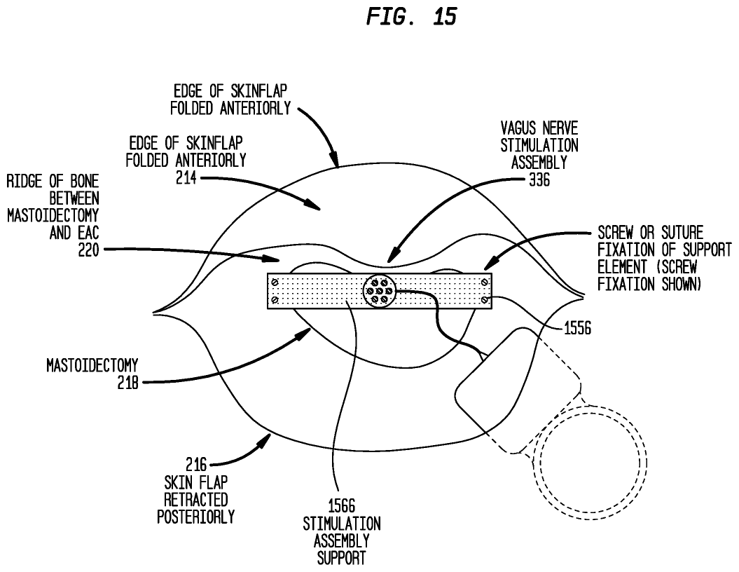

As noted above, an implantable vagal nerve stimulator in accordance with embodiments presented herein can be used as a stand-alone device or in combination with other implantable medical devices, such as cochlear implants, vestibular implants, etc. When used in a recipient who is being implanted with a cochlear implant or vestibular implant, the surgeon will form a mastoidectomy and location of the auricular branch of the vagal nerve dictates that the vagal nerve stimulation assembly may finally sit partly or fully over the cavity of the mastoidectomy. In certain such embodiments, it may be beneficial to provide support for the vagal nerve stimulation assembly so it does not separate from the tissue to be stimulated. FIG. 15 illustrates one example stimulation assembly support structure 1566 for use with the vagal nerve stimulation assembly 336, described above with reference to FIGS. 3A-3C and the line drawing 210 from FIG. 2A.

FIG. 15 illustrates that the stimulation assembly support structure 1566 is an elongate member having a length that is sufficient to extend across the mastoidectomy 218. As different surgeons form different sized mastoidectomies, the length of the stimulation assembly support structure 1566 can vary for different recipients. The stimulation assembly support structure 1566 is formed from a biocompatible material having sufficient rigidity to retain the vagal nerve stimulation assembly 336 adjacent to the soft tissues inclusive of the auricular branch of the vagal nerve (e.g., tissue flap 214), even though the vagal nerve stimulation assembly 336 is positioned over the mastoidectomy 218. The stimulation assembly support structure 1566 can be secured/fixed (e.g., via sutures, bone screws, etc.) within the recipient. In certain embodiments, the stimulation assembly support structure 1566 can include fixation points 1556) that facilitate fixation of the stimulation assembly support structure 1566 to the tissue/and/or bone. The vagal nerve stimulation assembly 336 can, in turn, be affixed to the stimulation assembly support structure 1566. The support structure 1566 could be made of a variety of materials including silicone, polymers, metals, alloys, or combinations of these materials.

In the embodiments of FIG. 15, stimulation assembly support structure 1566 could be a separate component or, in alternative embodiments, could be integrated with the vagal nerve stimulation assembly 336. In such embodiments, the stimulation assembly support structure 1566 integrated with the vagal nerve stimulation assembly 336 could be fixed in place and the tissue simply folded back over onto the electrode assembly. A combined stimulation assembly and support structure would have the advantage of avoiding dead space between the back of the stimulation assembly and the support structure.

As noted, the embodiments of FIG. 15 may be particularly useful in cases that a mastoidectomy is needed, such as when an implantable vagal nerve stimulator is combined with a cochlear implant or a vestibular implant. However, even in cases in which no mastoidectomy is needed (e.g., stand-alone implantable vagal nerve stimulator), the support structure 1566 may still benefit in this concept to fix the pad to the bone to simplify surgery.

As noted, the stimulation assemblies described herein can be well secured/stabilized in the recipient (e.g., on the bone or tissue) in an area of little movement compared to the neck. As such, the techniques presented herein are likely to have a high reliability compared to devices with electrodes in the neck which is highly mobile and more likely to stress the electrode lead and connection to the nerve.

In the descriptions above, the designs have generally been described with reference to combinations of vagal nerve stimulation with cochlear implants and/or vestibular implants where a mastoidectomy is required as part of the cochlear implant or vestibular implant surgery. In the case of a stand-alone implantable vagal nerve stimulator (e.g., stimulation of the auricular branch of the vagal nerve without a cochlear implant or vestibular implant), then a full mastoidectomy is most likely not required. This allows for addition alternatives for surgical approach and stimulation design. However, it is to be appreciated that any of the above techniques could also be used with a stand-alone implantable vagal nerve stimulator.

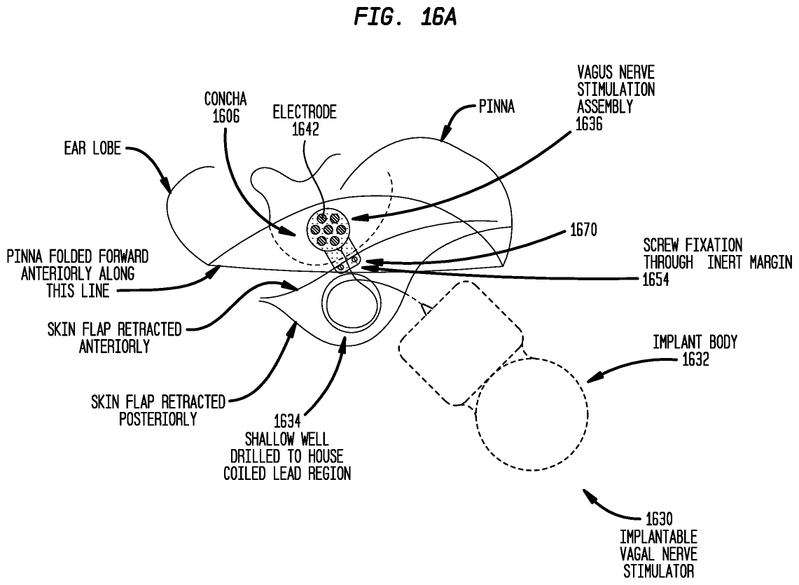

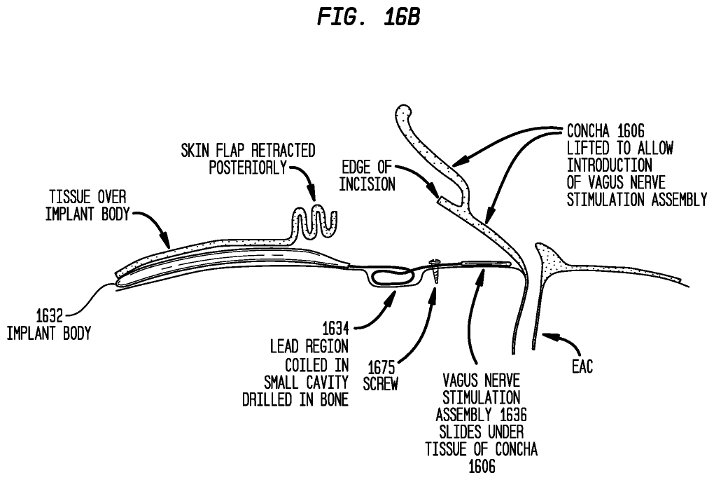

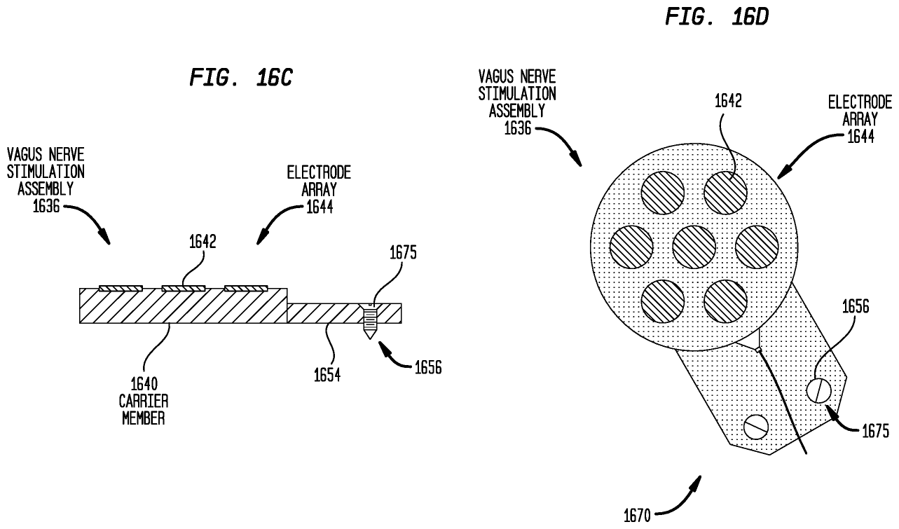

The surgical approach for a stand-alone implantable vagal nerve stimulator could be similar to that described above, or alternatively the surgeon could elect to make the incision closer to the site of the stimulation assembly and simply lift the tissue of the pinna and concha without folding it anteriorly. This may, for example, have the benefit of reducing the chance of damaging the finer threads of the A auricular branch of the vagal nerve and thus improving the stimulation sensitivity. FIGS. 16A, 16B, 16C, and 16D illustrate one such embodiment.

More specifically, FIG. 16A is a schematic top-view of an implantable vagal nerve stimulator 1630 during surgical placement (e.g., during surgery) of a vagal nerve stimulation assembly 1636, while FIG. 16B is a schematic side-view of the implantable vagal nerve stimulator 1630 during surgical placement of the vagal nerve stimulation assembly 1636. For ease of description, FIGS. 16A and 16B illustrate the implantable vagal nerve stimulator 1630 with a line drawing in which anatomical structures of a left ear of a recipient are shown in a simplified form. FIGS. 16C and 16D are cross-section and top-views, respectively of the vagal nerve stimulation assembly 1636. For ease of description, FIGS. 16A-16D will be described together.

As shown in FIGS. 3A-3C, the implantable vagal nerve stimulator 1630 comprises an implant body (main module) 1632, a lead region 1634, and the vagal nerve stimulation assembly 336, all configured to be implanted under the skin/tissue of the recipient. The implant body 1632 may be substantially similar to implant body 332 of FIG, 3A and, as such, is not described in detail.

As noted, vagal nerve stimulation assembly 1636 is configured to be implanted in the recipient. Vagal nerve stimulation assembly 1636 is formed by an electrically non-conductive (insulating) carrier member 1640 and a plurality of conductive electrodes 1642 disposed in (in or on) the carrier member 1640. The plurality of electrodes collectively form an electrode array 1644. In the examples of FIGS. 16A-16D, the vagal nerve stimulation assembly 1636 is implanted/placed by sliding the stimulation assembly under the concha 1606, where, as shown in FIG. 16B, the concha is lifted from a posterior incision to enable the vagal nerve stimulation assembly 1636 to be inserted. Once implanted, the plurality of electrodes 1642 face towards the concha 1606 (e.g., the tissue containing the auricular branch of the vagal nerve).

In this example, the posterior edge 1670 of the vagal nerve stimulation assembly 1636 is secured to the bone with, for example, sutures, an adhesive, surgical screws, etc. Similar to the above embodiments, the stimulation assembly 1636 includes features to facilitate fixation, namely an inert margin inert margin 1654 with fixation points 1656. As described above, the fixation points 1656 comprise integrated mechanical weaknesses in the carrier member material that enable a surgeon to secure the vagal nerve stimulation assembly 1636 within the recipient. The fixation points 1656 can comprise, for example, pre-formed apertures in the carrier member 1640, relatively thinner sections of the carrier member 1640, etc. In the embodiments of FIGS. 16A-16D, the vagal nerve stimulation assembly 1636 is secured to the recipient's bone via screws 1675, but other techniques can be used in alternative embodiments.

In the examples of FIGS. 16A-16D, the vagal nerve stimulation assembly 1636 could be sandwiched between the tissue and cortical bone and a shallow cavity can be drilled into the bone to provide space for the lead region 1634 to protect it from impact and prevent erosion and evulsion. This cavity would not need to be as deep or large as a full mastoidectomy.

FIGS. 16A-16D illustrate that the portion of the vagal nerve stimulation assembly 1636 in which the electrodes 1642 are disposed has a generally planar and circular shape. It is to be appreciated that the stimulation assembly 1636 could have alternative shapes and arrangements, including any of the arrangements described above.

In the embodiments of FIGS. 16A-16D, the vagal nerve stimulation assembly 1636 is fixed to prevent gradual migration of the stimulation assembly from the site of the auricular branch of the vagal nerve. Again, as noted, FIGS. 16A-16D illustrate one option for screw fixation, but other fixation options include suture fixation to the bone, use of surgical adhesives to the bone, etc. It would also be possible to suture to the tissue over the vagal nerve stimulation assembly 1636 to provide a stable fixation relative to the auricular branch of the vagal nerve embedded in the tissue. The design also provides slack in the lead region to prevent the possibility of a movement of the implant body 1632 from excerpt forces to pull the vagal nerve stimulation assembly 1636 out of position.

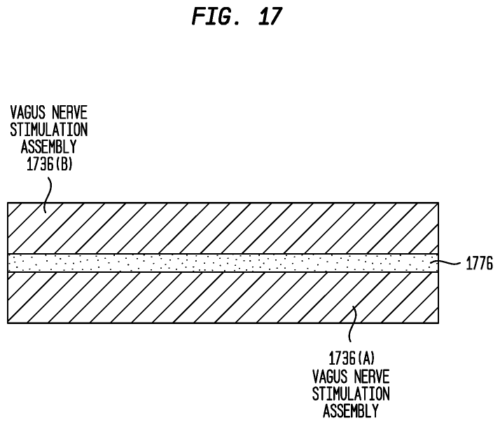

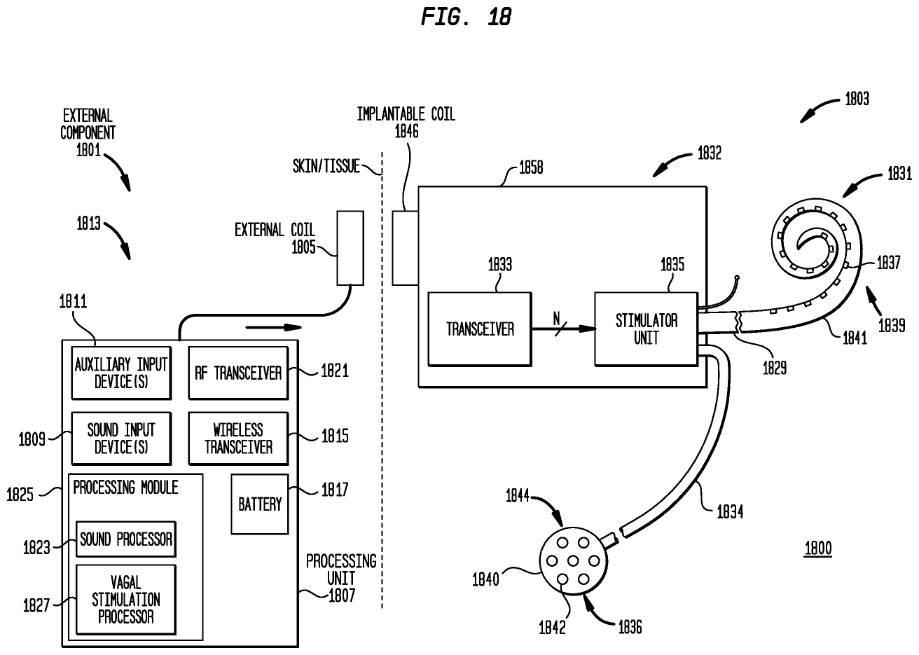

The above embodiments have primarily been described with reference to the implantation of a single vagal nerve stimulation assembly and use of that vagal nerve stimulation assembly to stimulate the auricular branch of the vagal nerve. In alternative embodiments, a plurality of vagal nerve stimulation assemblies can be implanted and collectively used to stimulate the auricular branch of the vagal nerve. One such example embodiment is shown in FIG. 17.

More specifically, as described elsewhere herein, the auricular branch of the vagal nerve is located within the tissue below the concha of the recipient. However, this tissue comprises a plurality of different tissue layers that can be surgically separated and the auricular branch of the vagal nerve is generally known to be positioned specifically in the periosteal “palva” flap. FIG. 17 is a schematic diagram illustrating an embodiment in which a first vagal nerve stimulation assembly 1736(A) is implanted on a first side of the palva flap 1776 and a second stimulation assembly 1736(B) is implanted on a second side of the palva flap 1776. The stimulation assembly 1736(A) and stimulation assembly 1736(B) could each have an arrangement similar to the other stimulation assemblies described herein, or another arrangement.

In the example of FIG. 17, the stimulation assemblies 1736(A) and 1736(B) deliver stimulation signals between one another. Since the auricular branch of the vagal nerve is located between the stimulation assemblies 1736(A) and 1736(B), the stimulation signals passing between the stimulation assemblies 1736(A) and 1736(B) stimulate the auricular branch of the vagal nerve. Due the close proximity of the electrodes in the stimulation assemblies 1736(A) and 1736(B), these embodiments can result in targeted stimulation of the auricular branch of the vagal nerve. In one example, stimulation assembly 1736(A) is implanted/positioned on the bone medial to the auricular branch of the vagal nerve and the stimulation assembly 1736(B) is implanted in the loose areolar tissue above the auricular branch of the vagal nerve so that a bipolar stimulation between the stimulation assemblies captures the auricular branch of the vagal nerve.

As noted above, placed at least one multi-electrode vagal nerve stimulation assembly into the recipient, an electrode selection process can be performed to activate and/or deactivate various combinations of the electrodes to optimally stimulate the auricular branch of the vagal nerve and/or to avoid stimulation of other nerves in the vicinity of the stimulation assembly.

In one example, the techniques presented herein include an exercise of checking thresholds on each of the electrodes. In an initial step, stimulation would be delivered via each electrode individually and the stimulation level (current) is progressively (e.g., incrementally) increased from a low level to a relatively higher level. By capturing objective or subjective feedback, this process could identify any electrodes that result in undesirable stimulation of other nerves. Any electrodes determined to result in undesirable stimulation could be deactivated (switched off), use stimulation levels reduced, and or use in other manners in which the undesirable stimulation does not occur.

In another step, the user (e.g., clinician, surgeon, etc.) may look for objective measures related to the target outcome for the stimulation. In the case of a combined cochlear implant and vagal nerve stimulator, the user could look for measures that indicate activation of the Locus Coeruleus. One such measure is a drop in pressure of oxygen (PO2). Another step would be to trial stimulation of the auricular branch of the vagal nerve and monitor progress by, for example, monitoring development of speech performance with the cochlear implant.

In another step, the user (e.g., clinician, surgeon, etc.) may look for objective measures related to the target outcome for the stimulation. In the case of a combined cochlear implant and vagal nerve stimulator, the user could look for measures that indicate activation of the Locus Coeruleus. One such measure is a drop in pressure of oxygen (PO2) Another step would be to trial stimulation of the auricular branch of the vagal nerve and monitor progress by, for example, monitoring development of speech performance with the cochlear implant. As noted above, another option is to provide the recipient with means to adjust stimulation based on their subjective determination of an optimal stimulation regime. This could include: choice of electrodes; stimulation levels; duration of stimulation; timing of stimulation (e.g. during the day or night; or whether at same time or different time to cochlear stimulation).

The techniques presented herein may be used to provide stand-alone stimulation of the vagal nerve for treatment of a variety of medical conditions and diseases. For example, stimulation of the vagal nerve can be useful in treating depression, epilepsy, memory reinforcement (e.g., to forestall impact of cognitive loss with aging), etc.

Additionally, the techniques presented herein may be used to provide potential enhancements to other types of implantable medical devices, such as cochlear implants or vestibular implants. For example, there may be advantages in providing a device that can stimulate the vagal nerve in combination with the cochlear nerve (e.g., combined cochlear implant and vagal nerve stimulator); a device that can stimulate the vagal nerve in combination with the vestibular organs (e.g., combined vestibular implant and vagal nerve stimulator), or all of the cochlear nerve, the vagal nerve and the vestibular organs (e.g., combined vestibular implant, vagal nerve stimulator, and vestibular implant). Other combinations of stimulators for various treatments are also envisaged including: a combined epilepsy monitor and vagal nerve stimulator, a combined epilepsy stimulator and vagal nerve stimulator, a combined tinnitus stimulator and vagal nerve stimulator, etc. Yet another potential application of vagal nerve stimulation is to provide an anti-inflammatory effect to assist with neural survival and/or preservation of hearing for people who have significant levels of low-frequency hearing and yet need a cochlear implant for high frequency stimulation. These cases are referred to as receiving electro-Acoustic stimulation (e.g., combined electro-acoustic hearing prosthesis and vagal nerve stimulator).

The techniques presented herein can be used to provide a device that offers a combination of therapies with one device and single surgery. For example, one of the larger populations with hearing diseases are older people. The older population tends to have multiple disorders, so a single device that can treat two diseases at once may be cost-effective and minimize risk by only requiring a single surgery.

FIG. 18 is a block diagram of an example combined cochlear implant and vagal nerve stimulator, in accordance with certain embodiments presented. For ease of reference, the combined cochlear implant and vagal nerve stimulator is referred to herein as a cochlear-vagal stimulator 1800.

In this example, the cochlear-vagal stimulator 1800 comprises an external component 1801 and an internal/implantable component 1803. The external component 1801 is directly or indirectly attached to the body of the recipient and typically comprises an external coil 1805 and, generally, a magnet (not shown in FIG. 18) fixed relative to the external coil 1805. The external component 1801 also comprises one or more input elements/devices 1813 for receiving input signals at a processing unit 1807. In this example, the one or more one or more input devices 1813 include sound input devices 1809 (e.g., microphones positioned by auricle of the recipient, telecoils, etc.) configured to capture/receive input signals, one or more auxiliary input devices 1811 (e.g., audio ports, such as a Direct Audio Input (DAI), data ports, such as a Universal Serial Bus (USB) port, cable port, etc.), and a wireless transmitter/receiver (transceiver) 1815, each located in, on, or near the processing unit 1807.

The sound processing unit 1807 also includes, for example, at least one battery 1817, a radio-frequency (RF) transceiver 1821, and a processing module 1825. The processing module 1825 comprises a number of elements, including a sound processor 1823, and a vagal stimulation processor 1827. Each of the sound processor 1823 and the vagal stimulation processor 1827 may be formed by one or more processors (e.g., one or more Digital Signal Processors (DSPs), one or more uC cores, etc.), firmware, software, etc. arranged to perform operations described herein. That is, the sound processor 1823 and the vagal stimulation processor 1827 may each be implemented as firmware elements, partially or fully implemented with digital logic gates in one or more application-specific integrated circuits (ASICs), partially or fully in software, etc.

In the examples of FIG. 18, the processing unit 1807 can be, for example, a behind-the-ear (BTE) unit (e.g., a component configured to be attached to, and worn adjacent to, the recipient's ear), an off-the-ear (OTE) unit (e.g., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

Returning to the example embodiment of FIG. 18, the implantable component 1803 comprises an implant body (main module) 1832, a cochlear lead region 1829, an intra-cochlear stimulating assembly 1831, a vagal lead region 1834, and a vagal nerve stimulation assembly 1836, all configured to be implanted under the skin/tissue (tissue) of the recipient. The implant body 1832 generally comprises a hermetically-sealed housing 1838 in which RF interface circuitry 1833 and a stimulator unit 1835 are disposed. The implant body 1832 also includes an internal/implantable coil 1846 that is generally external to the housing 1838, but which is connected to the RF interface circuitry 1833 via a hermetic feedthrough (not shown in FIG. 18).

The intra-cochlear stimulating assembly 1831 is configured to be at least partially implanted in the recipient's cochlea. The intra-cochlear stimulating assembly 1831 includes a plurality of longitudinally spaced intra-cochlear electrical stimulating contacts (electrodes) 1837 that collectively form a contact or electrode array 1839 for delivery of electrical stimulation (current) to the recipient's cochlea. The electrode array 1839 is disposed in/on a carrier member 1841. Stimulating assembly 1831 extends through an opening in the recipient's cochlea (e.g., cochleostomy, the round window, etc.) and has a proximal end connected to stimulator unit 1835 via cochlear lead region 1829 and a hermetic feedthrough (not shown in FIG. 18). Lead region 1829 includes a plurality of conductors (wires) that electrically couple the electrodes 1837 to the stimulator unit 1835.

As described elsewhere herein, the vagal nerve stimulation assembly 1836 is configured to be implanted adjacent the recipient's concha. The vagal nerve stimulation assembly 1836 includes a plurality of electrodes 1842 that collectively form a contact or electrode array 1844 for delivery of electrical stimulation (current) to the auricular branch of the vagal nerve. The electrode array 1844 is disposed in/on a carrier member 1840. Stimulating assembly is electrically connected to the stimulator unit 1835 via vagal lead region 1834 and a hermetic feedthrough (not shown in FIG. 18). Lead region 1834 includes a plurality of conductors (wires) that electrically couple the electrodes 1842 to the stimulator unit 1835.

As noted, the cochlear-vagal stimulator 1800 includes the external coil 1805 and the implantable coil 1846. The coils 1805 and 1846 are wire antenna coils each comprised of multiple turns of electrically insulated single-strand or multi-strand platinum or gold wire. Generally, a magnet is fixed relative to each of the coils 1805 and 1846, where the magnets facilitate the operational alignment of the external coil with the implantable coil. This operational alignment of the coils 1805 and 1846 are enables the external component 1801 to transmit power and/or data to the implantable component 1803 via a closely-coupled wireless link formed between the coils 1805 and 1846. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 18 illustrates only one example arrangement.

As noted above, the processing module 1825 includes the sound processor 1823 and the vagal stimulation processor 1827. The sound processor 1823 is configured to control the cochlear stimulation operations. The vagal stimulation processor 1827, in turn, is configured to control the vagal nerve stimulation operations. In operation, the sound processor 1823 and the vagal stimulation processor 1827 each generate control signals that are provided to the implantable component 1803 via the closely-coupled wireless link formed between the coils 1805 and 1846. These control signals are used by the stimulator unit 1835 to stimulator the recipient's cochlear and/or the auricular branch of the vagal nerve.

FIG. 18 illustrate an arrangement in which the cochlear-vagal stimulator 1800 includes an external component. However, it is to be appreciated that embodiments of the present invention may be implemented in cochlear implants having alternative arrangements. For example, in an alternative embodiment, all components of a cochlear-vagal stimulator could be configured to be implanted under skin/tissue of a recipient. Because all components are implantable, such a cochlear-vagal stimulator operates, for at least a finite period of time, without the need of an external device. An external device can be used to, for example, charge an internal power source (battery) of the cochlear-vagal stimulator.

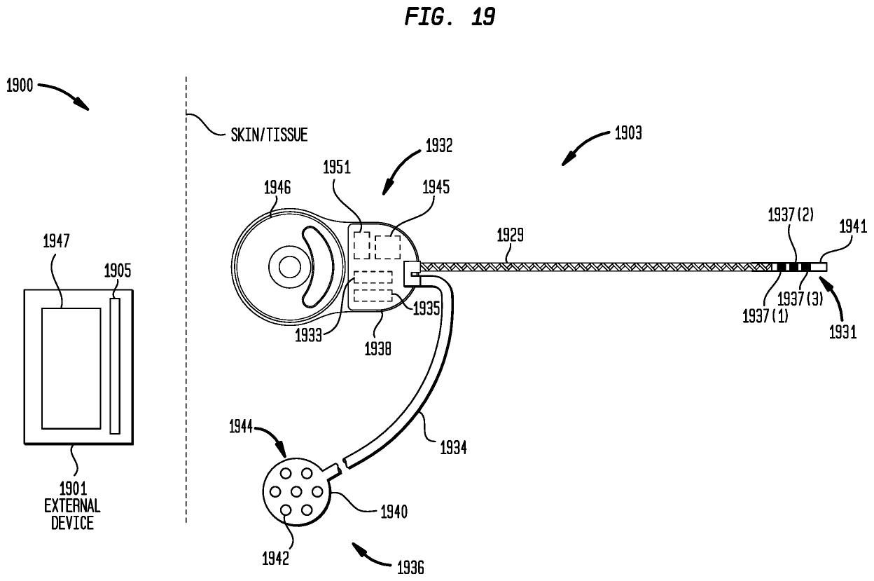

FIG. 19 is a block diagram of an example combined vestibular implant and vagal nerve stimulator, in accordance with certain embodiments presented. For ease of reference, the combined vestibular implant and vagal nerve stimulator is referred to herein as a vestibular-vagal stimulator 1900.

In this example, the vestibular-vagal stimulator 1900 comprises an implantable component 1903 and an external device/component 1901. More specifically, the implantable component 1903 comprises an implant body (main module) 1932, a vestibular lead region 1929, an vestibular stimulating assembly 1931, a vagal lead region 1934, and a vagal nerve stimulation assembly 1936, all configured to be implanted under the skin/tissue (tissue) of the recipient. The implant body 1932 generally comprises a hermetically-sealed housing 1838 in which RF interface circuitry 1933, a stimulator unit 1935, one or more rechargeable batteries 1945, and one or more processors 1951 are disposed. The implant body 1932 also includes an internal/implantable coil 1946 that is generally external to the housing 1938, but which is connected to the RF interface circuitry 1933 via a hermetic feedthrough (not shown in FIG. 19).

The vestibular stimulating assembly 1931 comprises a plurality of electrodes 1937 disposed in a carrier member (e.g., a flexible silicone body) 1941. In this specific example, the stimulating assembly 1931 comprises three (3) stimulation electrodes, referred to as electrodes 1937(1), 1937(2), and 1937(3). The electrodes 1937(1), 1937(2), and 1937(3) function as an electrical interface for delivery of electrical stimulation signals to the recipient's vestibular system. Lead region 1929 includes a plurality of conductors (wires) that electrically couple the electrodes 1937(1), 1937(2), and 1937(3) to the stimulator unit 1935.

The stimulating assembly 1931 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

As described elsewhere herein, the vagal nerve stimulation assembly 1936 is configured to be implanted adjacent the recipient's concha. The vagal nerve stimulation assembly 1936 includes a plurality of electrodes 1942 that collectively form a contact or electrode array 1944 for delivery of electrical stimulation (current) to the auricular branch of the vagal nerve. The electrode array 1944 is disposed in/on a carrier member 1940. Stimulating assembly 1936 is electrically connected to the stimulator unit 1935 via vagal lead region 1934 and a hermetic feedthrough (not shown in FIG. 19). Lead region 1934 includes a plurality of conductors (wires) that electrically couple the electrodes 1942 to the stimulator unit 1935. The one or more and one or more processors 1951 are configured to control the delivery of stimulation signals to the vestibular system and/or the auricular branch of the vagal nerve via the stimulation assembly 1931 and 1936, respectively.

As noted above, the implant body 1932 comprises RF interface circuitry 1933 and one or more rechargeable batteries 1945. In certain examples, the external device 1901 is configured to charge/recharge the one or more rechargeable batteries through the inductive transfer of power via the RF interface circuitry 1933. That is, the external device 1901 comprises an external coil 1905 configured to be inductively coupled with the implantable coil 1946. When inductively coupled, the external coil 1905 and the implantable coil 1946 form a closely-coupled wireless link by which power is transferred from one or more rechargeable batteries 1947 of the external device 1901 through the skin/tissue of the recipient to the RF interface circuitry 1933.

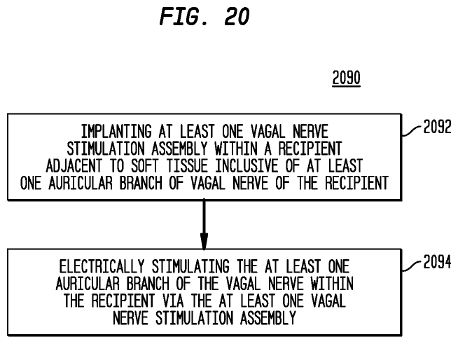

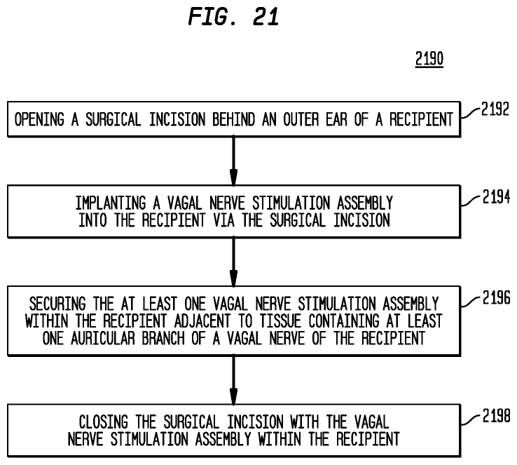

FIG. 20 is a flowchart of an example method 2090, in accordance with embodiments presented herein. Method 2090 beings at 2092 where at least one vagal nerve stimulation assembly is positioned/implanted within a recipient adjacent to soft tissues inclusive of the auricular branch of the vagal nerve. At 2094, at least one auricular branch of a vagal nerve of the recipient is electrically stimulated via the least one vagal nerve stimulation assembly. In certain embodiments, the method includes stimulating a trunk of the auricular branch of the vagal nerve, while in other embodiments method includes stimulating one or more afferent branches of the auricular branch of the vagal nerve. In certain embodiments, the method includes stimulating both the trunk and one or more afferent branches of the auricular branch of the vagal nerve.

FIG. 21 is a flowchart of an example method 2190, in accordance with embodiments presented herein. Method 2190 beings at 2192 where a surgeon opens a surgical incision behind an outer ear of a recipient. At 2194, the surgeon implants a vagal nerve stimulation assembly into the recipient via the surgical incision. At 2196, the surgeon secures the vagal nerve stimulation assembly within the recipient adjacent to tissue containing at least one auricular branch of the vagal nerve of the recipient . At 2198, the surgeon closes the surgical incision with the vagal nerve stimulation assembly within the recipient.

The following description provides surgical considerations related to implantation of an vagal nerve stimulation assembly, in accordance with certain embodiments presented herein.

### Postauricular Incision

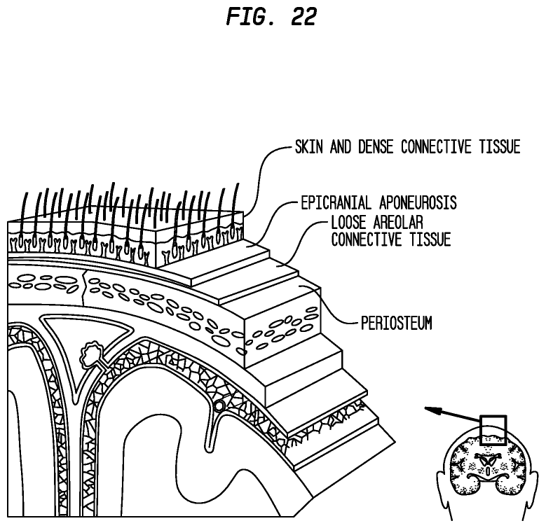

After administration of, for example, 1:100,000 lidocaine/epinephrine at incision site, it is recommended that the surgeon pause surgical efforts for a full five minutes before making an incision. This will mitigate amount of bleeding and, thus, less need for cautery. Five minutes is the accepted amount of time for potency of epinephrine.
It is recommended to use a “cold steel” scalpel to incise soft tissue (e.g., 15 Blade is commonly used)
It is recommended that monopolar cautery not be used and that bipolar cautery is used only as necessary. Additionally, a cauterizing scalpel would be contraindicated under this guidance.
The surgeon must take care to dissect down to level of loose areolar tissue just above periosteum to limit damaging nerve fibers (e.g., it is important not to cut the vagus nerve and not to damage the when pulling back the tissue). See FIG. 22 for reference of main soft tissue layers.
When freeing soft tissue from postauricular incision anteriorly and posteriorly, prior to incising/creating palva flap, it is recommended to use blunt dissection in the avascular plane just lateral to level of periosteum to create soft tissue flap above the level of periosteum. This refers to separating the “SCAL” layers (e.g., Skin (S), Dense Connective Tissue (C), Epicranial Aponeurosis (A), and Loose areolar connective tissue (L)) shown in FIG. 22, while leaving the periosteum (P) in place. It is noted that use of sharp/traumatic dissection may inadvertently damage nerve fibers.

### Creation of Fibromuscular/Periosteal “Palva” Flap

It is recommended not utilize a “T-shaped” incision or linear-type to gain access to mastoidectomy site.
Anteriorly-based flap may be used to minimize chances of damaging nerve elements.
It is recommended to make the incision through the periosteum with the “cold steel” scalpel in a wide footprint to as much fibrous structure is left of vagus nerve elements.
It is recommended to consider use of wider Lempert Elevator rather than curved/rounded Freer Elevator to lift periosteal flap. Use of this type of elevator will mitigate chances of damaging/stretching nerve fibers during the process of separating periosteal palva from the cortical bone surface. If that specific type of elevator is not available, take care during this “freeing” process to protect nerve elements.

### Addressing Palva Flap After its Creation

It is recommended not to readjust self-retaining retractors to include palva flap in retracted tissues. The palva flap should be free from the grip of the retractor. A self-retaining retractor (typically a Weitlaner retractor) may cause damage to nerve elements. Consider use of hook retraction or other method so as to not apply force directly to nerve elements.
Surgical clamps utilized in other regions/markets should be used with caution and care.
It is recommended to place a single layer of thin surgical towel between periosteum and retracted soft tissue as well as additional single layer of same towel material on lateral side and flap with additional single layer of same towel on lateral side to mitigate changes of strike-damage from high speed bur, bone dust encroachment and temperature fluctuations, etc.
It is recommended to not wrap the palva flap in a towel, as strike from a bur would pull towel into bur with the flap still inside.
The palva flap (with protective towels) should lay gently on side of retractor arms to apply no force on this flap.
To a predetermined amount of time, systematically wet the towels/flap. Wetting of towels/flap should be with body-temperature water.
It is recommended to consider drip-irrigation during course of surgery so long as field of work does not require “dry” environment. The wetting mixture should have a predetermined amount of steroid dilution to mitigate damage to nerve elements.

### Addressing Auricular Branch of Vagus Nerve During Case (ABVN)

Live monitoring may be beneficial to ascertain damage/health of the nerve in method comparable to facial nerve monitoring systems utilized on cochlear implant procedures (e.g., monitor the vagus nerve to monitor whether the vagus nerve is being stimulated by physical or electrical insult). Probes would be placed during prep/drape of patient and live monitoring would continue throughout the case.
Ancillary probe monitoring/testing may be beneficial to locate correct branch of the ABVN (e.g., stimulate the vagus nerve through the implant during surgery to check that the vagus electrode is properly placed and making good contact with the nerve). A secondary probe will be connected to the nerve monitoring system and will give stimulated location/distance of branch bundle to focus stimulation on.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A method, comprising:
implanting at least one vagal nerve stimulation assembly within a recipient adjacent to soft tissue inclusive of at least one auricular branch of a vagal nerve of the recipient; and
electrically stimulating the at least one auricular branch of the vagal nerve within the recipient via the at least one vagal nerve stimulation assembly.

2. The method of claim 1, wherein electrically stimulating the at least one auricular branch of the vagal nerve within the recipient comprises:
electrically stimulating a trunk of the at least one auricular branch of the vagal nerve within the recipient.

3. The method of claim 1, wherein electrically stimulating the at least one auricular branch of the vagal nerve within the recipient comprises:
electrically stimulating one or more afferent branches of the at least one auricular branch of the vagal nerve within the recipient.

4. The method of claim 1, wherein the at least one vagal nerve stimulation assembly comprises one or more electrodes disposed on a first surface of a carrier member, and wherein implanting the at least one vagal nerve stimulation assembly within the recipient adjacent to soft tissue inclusive of the at least one auricular branch of the vagal nerve comprises:
positioning the at least one vagal nerve stimulation assembly so that, when implanted, the one or more electrodes face away from a skull bone of the recipient and towards an underside of the soft tissue inclusive of the at least one auricular branch of the vagal nerve.

5. The method of claim 1, further comprising:
implanting an at least second vagal nerve stimulation assembly adjacent to soft tissue inclusive of the at least one auricular branch of the vagal nerve.

6. The method of claim 5, wherein electrically stimulating the at least one auricular branch of the vagal nerve via the at least one vagal nerve stimulation assembly comprises:
delivering electrical stimulation signals between the at least one vagal nerve stimulation assembly and the at least second vagal nerve stimulation assembly.

7. The method of claim 6, wherein the at least one vagal nerve stimulation assembly is implanted adjacent a skull bone medial to the at least one auricular branch of the vagal nerve and the at least second vagal nerve stimulation assembly is implanted in loose areolar tissue above the at least one auricular branch of the vagal nerve.

8. The method of claim 1, wherein the at least one vagal nerve stimulation assembly comprises one or more electrodes, and wherein electrically stimulating the at least one auricular branch of the vagal nerve via the at least one vagal nerve stimulation assembly comprises:
delivering electrical stimulation signals between at least one of the one or more electrodes and a ground electrode that is separate from the at least one vagal nerve stimulation assembly.

9. The method of claim 1 wherein the at least one vagal nerve stimulation assembly comprises a plurality of electrodes, and wherein electrically stimulating the at least one auricular branch of a vagal nerve via the at least one vagal nerve stimulation assembly comprises:
delivering electrical stimulation signals between two or more of the plurality of electrodes of the at least one vagal nerve stimulation assembly.

10. The method of claim 1, further comprising:
securing the at least one vagal nerve stimulation assembly within the recipient adjacent to the soft tissue inclusive of the at least one auricular branch of the vagal nerve.

11. (canceled)

12. The method of claim 10, wherein securing the at least one vagal nerve stimulation assembly within the recipient adjacent to the soft tissue inclusive of the at least one auricular branch of the vagal nerve comprises:
at least one of adhering, suturing, or screwing the at least one vagal nerve stimulation assembly to at least one of tissue or bone of the recipient.

13. (canceled)

14. The method of claim 1, wherein the at least one vagal nerve stimulation assembly comprises a plurality of electrodes, and wherein the method comprises:
selecting a subset of the plurality of electrodes for use in electrically stimulating the at least one auricular branch of the vagal nerve; and
electrically stimulating the at least one auricular branch of the vagal nerve using only the subset of the plurality of electrodes.

15. An apparatus, comprising:
at least one stimulation assembly configured to be implanted within a recipient adjacent to a distal surface of soft tissue inclusive of at least one auricular branch of a vagal nerve of the recipient, wherein the at least one stimulation assembly comprises one or more electrodes facing the distal surface of the soft tissue inclusive of at least one auricular branch of the vagal nerve;
an implantable module comprising a stimulator unit; and
a lead region electrically connecting the stimulator unit to the one or more electrodes.

16. The apparatus of claim 15, wherein the stimulator unit is configured to electrically stimulate at least one auricular branch of a vagal nerve of the recipient via the one or more electrodes.

17. The apparatus of claim 15, wherein the at least one stimulation assembly comprises an electrically non-conductive carrier member having a first surface and a second surface, and wherein the one or more electrodes are disposed on the first surface of the carrier member.

18. The apparatus of claim 17, wherein the carrier member comprises a plurality of integrated mechanical weaknesses in the carrier member that enable a surgeon to at least one of suture or screw the carrier member to tissue or bone of the recipient.

19. The apparatus of claim 18, wherein the plurality of integrated mechanical weaknesses comprise pre-formed apertures in the carrier member.

20. The apparatus of claim 18, wherein the plurality of integrated mechanical weaknesses comprise relatively thinner sections of the carrier member.

21. The apparatus of claim 17, wherein the first surface of the carrier member comprises a generally planar surface with a circular shape.

22. The apparatus of claim 17, wherein the first surface of the carrier member comprises an elongate planar surface.

23. The apparatus of claim 22, wherein the one or more electrodes comprises a plurality of electrodes disposed in at least one row.

24. The apparatus of claim 23, wherein the a least one row comprises a plurality of parallel rows.

25. The apparatus of claim 17, wherein the carrier member is formed from an resilient material that allows the carrier member to expand in a longitudinal direction.

26. The apparatus of claim 15, further comprising:
a stimulation assembly support structure configured to be implanted in the recipient between the at least one stimulation assembly and a skull bone of the recipient.

27-38. (canceled)
